SEQUENCE LISTING (138 sequences)

SEQ ID NO: 1 - DNA, 20 bp, Artificial Sequence
  Forward primer
cccagagctgcaagagctac

SEQ ID NO: 2 - DNA, 22 bp, Artificial Sequence
  Forward primer
gccgtgccgctctatctgtgag

SEQ ID NO: 3 - DNA, 28 bp, Artificial Sequence
  Forward primer
ataggcctgatcatctgaatctccttca

SEQ ID NO: 4 - DNA, 28 bp, Artificial Sequence
  Forward primer
aacctcggctacaacgtgagaccaagat

SEQ ID NO: 5 - DNA, 22 bp, Artificial Sequence
  Forward primer
atcggctacaacctgacgcaca

SEQ ID NO: 6 - DNA, 28 bp, Artificial Sequence
  Forward primer
tctggaatgttcaccaaacattgaaact

SEQ ID NO: 7 - DNA, 25 bp, Artificial Sequence
  Forward primer
ctcatgaacaagttcggcttccagt

SEQ ID NO: 8 - DNA, 27 bp, Artificial Sequence
  Forward primer
gatgaggatgagagtgaggtgacatcc

SEQ ID NO: 9 - DNA, 18 bp, Artificial Sequence
  Forward primer
cacgcgctgtgcatggag

SEQ ID NO: 10 - DNA, 19 bp, Artificial Sequence
  Forward primer
catggaggcgcccaacaac

SEQ ID NO: 11 - DNA, 20 bp, Artificial Sequence
  Reverse primer
cacgatcagcgtcataaggt

SEQ ID NO: 12 - DNA, 18 bp, Artificial Sequence
  Reverse primer
gtggcgcgggaagtgctc

SEQ ID NO: 13 - DNA, 28 bp, Artificial Sequence
  Reverse primer
tcttggcacatcctcaaggtaataggtt

SEQ ID NO: 14 - DNA, 27 bp, Artificial Sequence
  Reverse primer
gtactggatgagcggtgtgaaagttgt

SEQ ID NO: 15 - DNA, 27 bp, Artificial Sequence
  Reverse primer
atgggcgtgtacatagtgcataggaag

SEQ ID NO: 16 - DNA, 28 bp, Artificial Sequence
  Reverse primer
tttctcataaagtttacgacaaggtgga

SEQ ID NO: 17 - DNA, 20 bp, Artificial Sequence
  Reverse primer
cgcggtagggtaggcagtgg

SEQ ID NO: 18 - DNA, 25 bp, Artificial Sequence
  Reverse primer
actcagacttcctggctctcaggtg

SEQ ID NO: 19 - DNA, 27 bp, Artificial Sequence
  Reverse primer
ggctcttctccacgtactggaacttct

SEQ ID NO: 20 - DNA, 20 bp, Artificial Sequence
  Reverse primer
gtccttcagcgggtgctcct

SEQ ID NO: 21 - DNA, 24 bp, Artificial Sequence
  FZD2 primer (reverse)
cagcgtcttgcccgaccagatcca

SEQ ID NO: 22 - DNA, 24 bp, Artificial Sequence
  FZD2 primer (forward)
ctagcgccgctcttcgtgtacctg

SEQ ID NO: 23 - DNA, 21 bp, Artificial Sequence
  FZD 5 primer (forward)
ttcatgtgcctggtggtgggc

SEQ ID NO: 24 - DNA, 21 bp, Artificial Sequence
  FZD5 primer (reverse)
tacacgtgcgacagggacacc

SEQ ID NO: 25 - DNA, 20 bp, Artificial Sequence
  G3PDH primer (forward)
accacagtccatgccatcac

SEQ ID NO: 26 - DNA, 20 bp, Artificial Sequence
  G3PDH primer (reverse)
tacagcaacagggtggtgga

SEQ ID NO: 27 - protein, 75 aa, Artificial Sequence
  pFZD2-TT
MCVGQNHSEDGAPALLTTAPPPGLQPGAGGTPGGPGGGGAPPRYATLEHPFHCGPSLVDDALINSTKIYSYFPSV

SEQ ID NO: 28 - DNA, 228 bp, Artificial Sequence
  Coding region for pFZD2-TT
atgtgcgtcggccagaaccactccgaggacggagctcccgcgctactcaccaccgcgccgccgccgggactgcagccgggtgccgggggcaccccgggtggcccgggcggcggcggcgctcccccgcgctacgccacgctggagcaccccttccactgcggccccagcctggtggacgacgccctgatcaacagcaccaagatctacagctactttcccagcgtgtag

SEQ ID NO: 29 - protein, 75 aa, Artificial Sequence
  pTT-FZD2
MVDDALINSTKIYSYFPSVGPSLCVGQNHSEDGAPALLTTAPPPGLQPGAGGTPGGPGGGGAPPRYATLEHPFHC

SEQ ID NO: 30 - DNA, 228 bp, Artificial Sequence
  Coding region for pTT-FZD2
atggtggacgacgccctgatcaacagcaccaagatctacagctactttcccagcgtgggccccagcctgtgcgtcggccagaaccactccgaggacggagctcccgcgctactcaccaccgcgccgccgccgggactgcagccgggtgccgggggcaccccgggtggcccgggcggcggcggcgctcccccgcgctacgccacgctggagcaccccttccactgctag

SEQ ID NO: 31 - protein, 75 aa, Artificial sequence
  PFZD2-MMVF
MCVGQNHSEDGAPALLTTAPPPGLQPGAGGTPGGPGGGGAPPRYATLEHPFHCGPSLKLLSLIKGVIVHRLEGVE

SEQ ID NO: 32 - DNA, 228 bp, Artificial Sequence
  Coding region for PFZD2-MMVF
atgtgcgtcggccagaaccactccgaggacggagctcccgcgctactcaccaccgcgccgccgccgggactgcagccgggtgccgggggcaccccgggtggcccgggcggcggcggcgctcccccgcgctacgccacgctggagcaccccttccactgcggccccagcctgaagctgctgagcctgatcaagggcgtgatcgtgcaccgcctggagggcgtggagtag

SEQ ID NO: 33 - protein, 75 aa, Artificial Sequence
  PMMVF-FZD2
MKLLSLIKGVIVHRLEGVEGPSLCVGQNHSEDGAPALLTTAPPPGLQPGAGGTPGGPGGGGAPPRYATLEHPFHC

SEQ ID NO: 34 - DNA, 228 bp, Artificial Sequence
  Coding region for PMMVF-FZD2
atgaagctgctgagcctgatcaagggcgtgatcgtgcaccgcctggagggcgtggagggccccagcctgtgcgtcggccagaaccactccgaggacggagctcccgcgctactcaccaccgcgccgccgccgggactgcagccgggtgccgggggcaccccgggtggcccgggcggcggcggcgctcccccgcgctacgccacgctggagcaccccttccactgctag

SEQ ID NO: 35 - protein, 517 aa, Mouse
MAVSWIVFDLWLLTVFLGQIGGHSLFSCEPITLRMCQDLPYNTTFMPNLLNHYDQQTAALAMEPFHPMVNLDCSRDFRPFLCALYAPICMEYGRVTLPCRRLCQRAYSECSKLMEMFGVPWPEDMECSRFPDCDEPYPRLVDLNLVGDPTEYSFLHVRDCSPPCPNMYFRREELSFARYFIGLISIICLSATLFTFLTFLIDVTRFRYPERPIIFYAVCYMMVSLIFFIGFLLEDRVACNASSPAQYKASTVTQGSHNKACTMLFMVLYFFTMAGSVWWVILTITWFLAAVPKWGSEAIEKKALLFHASAWGIPGTLTIILLAMNKIEGDNISGVCFVGLYDVDALRYFVLAPLCLYVVVGVSLLLAGIISLNRVRIEIPLEKENQDKLVKFMIRIGVFSILYLVPLLVVIGCYFYEQAYRGIWETTWIQERCREYHIPCPYQVTQMSRPDLILFLMKYLMALIVGIPSIFWVGSKKTCFEWASFFHGRRKKEIVNESRQVLQEPDFAQSLLRDPNT

SEQ ID NO: 36 - protein, 500 aa, Mouse
MAWPGTGPSSRGAPGGVGLRLGLLLQFLLLLRPTLGFGDEEERRCDPIRIAMCQNLGYNVTKMPNLVGHELQTDAELQLTTFTPLIQYGCSSQLQFFLCSVYVPMCTEKINIPIGPCGGMCLSVKRRCEPVLREFGFAWPDTLNCSKFPPQNDHNHMCMEGPGDEEVPLPHKTPLNCVLKCGYDAGLYSRSAKEFTDIWMAVWASLCFISTTFTVLTFLIDSSRFSYPERPIIFLSMCYNIYSIAYIVRLTVGRERISCDFEEAAEPVLIQEGLKNTGCAIIFLLMYFFGMASSIWWVILTLTWFLAAGLKWGHEAIEMHSSYFHIAAWAIPAVKTIVILIMRLVDADELTGLCYVGNQNLDALTGFVVAPLFTYLVIGTLFIAAGLVALFKIRSNLQKDGTKTDKLERLMVKIGVFSVLYTVPATCVIACYFYEISNWALFRYSADDSNMAVEMLKIFMSLLVGITSGMWIWSAKTLHTWQKCSNRLVNSGKVKREKRG

SEQ ID NO: 37 - protein, 599 aa, Mouse
MEWGYLLEVTSLLAALAVLQRSSGAAAASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYNRTDLTTAAPSPPRRLPPPPPPGEQPPSGSGHSRPPGARPPHRGGSSRGSGDAAAAPPSRGGKTGQIANCALPCHNPFFSQDERAFTVFWIGLWSVLCFVSTFATVSTFLIDMERFKYPERPIIFLSACYLFVSVGYLVRLVAGHEKVACSGGAPGAGGRGGAGGAAAAGAGAAGRGASSPGARGEYEELGAVEQHVRYETTGPALCTVVFLLVYFFGMASSIWWVILSLTWFLAAGMKWGNEAIAGYSQYFHLAAWLVPSVKSIAVLALSSVDGDPVAGICYVGNQSLDNLRGFVLAPLVIYLFIGTMFLLAGFVSLFRIRSVIKQQGGPTKTHKLEKLMIRLGLFTVLYTVPAAVVVACLFYEQHNRPRWEATHNCPCLRDLQPDQARRPDYAVFMLKYFMCLVVGITSGVWVWSGKTLESWRALCTRCCWASKGAAVGAGAGGSG

SEQ ID NO: 38 - protein, 516 aa, Homo sapiens
MARPDPSAPPSLLLLLLAQLVGRAAAASKAPVCQEITVPMCRGIGYNLTHMPNQFNHDTQDEAGLEVHQFWPLVEIQCSPDLRFFLCTMYTPICLPDYHKPLPPCRSVCERAKAGCSPLMRQYGFAWPERMSCDRLPVLGRDAEVLCMDYNRSEATTAPPRPFPAKPTLPGPPGAPASGGRTGQVPNCAVPCYQPSFSADERTFATFWIGLWSVLCFISTSTTVATFLIDMDTFRYPERPIIFLSACYLCVSLGFLVRLVVGHASVACSREHNHIHYETTGPALCTIVFLLVYFFGMASSIWWVILSLTWFLAAAMKWGNEAIAGYGQYFHLAAWLIPSVKSITALALSSVDGDPVAGICYVGNQNLNSLRRFVLGPLVLYLLVGTLFLLAGFVSLFRIRSVIKQGGTKTDKLEKLMIRIGIFTLLYTVPASIVVACYLYEQHYRESWEAALTCACPGHDTGQPRAKPEYWVLMLKYFMCLVVGITSGVWIWSGKTVESWRRFTSRCCCRPRRGHK

SEQ ID NO: 39 - protein, 533 aa, Homo sapiens
MAVAPLRGALLLWQLLAAGGAALEIGRFDPERGRGAAPCQAVEIPMCRGIGYNLTRMPNLLGHTSQGEAAAELAEFAPLVQYGCHSHLRFFLCSLYAPMCTDQVSTPIPACRPMCEQARLRCAPIMEQFNFGWPDSLDCARLPTRNDPHALCMEAPENATAGPAEPHKGLGMLPVAPRPARPPGRSCAPRCGPGVEVFWSRRDKDFALVWMAVWSALCFFSTAFTVLTFLLEPHRFQYPERPIIFLSMCYNVYSLAFLIRAVAGAQSVACDQEAGALYVIQEGLENTGCTLVFLLLYYFGMASSLWWVVLTLTWFLAAGKKWGHEAIEAHGSYFHMAAWGLPALKTIVILTLRKVAGDELTGLCYVASTDAAALTGFVLVPLSGYLVLGSSFLLTGFVALFHIRKIMKTGGTNTEKLEKLMVKIGVFSILYTVPATCVIVCYVYERLNMDFWRLRATEQPCAAAAGPGGRRDCSLPGGSVPTVAVFMLKIFMSLVVGITSGVWVWSSKTFQTWQSLCYRKIAAGRARAKACRA

SEQ ID NO: 40 - protein, 544 aa, Rat
LEAPLLLGVRAQPAGQVSGPGQQRPPPPQPQQGGQQYNGERGISIPDHGYCQPISIPLCTDIAYNQTIMPNLLGHTNQEDAGLEVHQFYPLVKVQCSAELKFFLCSMYAPVCTVLEQALPPCRSLCERAQGCEALMNKFGFQWPDTLKCEKFPVHGAGELCVGQNTSDKGTPTPSLLPEFWTSNPQHGLGEKDCGAPCEPTKVYGLMYFGPEELRFSRTWIGIWSVLCCASTLFTVLTYLVDMRRFSYPERPIIFLSGCYTAVAVAYIAGFLLEDRVVCNDKFAEDGARTVAQGTKKEGCTILFMMLYFFSMASSIWWVILSLTWFLAAGMKWGHEAIEANSQYFHLAAWAVPAIKTITILALGQVDGDVLSGVCFVGLNNVDALRGFVLAPLFVYLFIGTSFLLAGFVSLFRIRTIMKHDGTKTEKLEKLMVRIGVFSVLYTVPATIVIACYFYEQAFRDQWERSWVAQSCKSYAIPCPHLQGGGGVPPHPPMSPDFTVFMIKYLMTLIVGITSGFWIWSGKTLNSWRKFYTRLTNSKQGETT

SEQ ID NO: 41 - protein, 529 aa, Rat
MRARSALPRSALPRLLLPLLLLPAAGPAQFHGEKGISIPDHGFCQPISIPLCTDIAYNQTIMPNLLGHTNQEDAGLEVHQFYPLVKVQCSPELRFFLCSMYAPVCTVLEQAIPPCRSICERARQGCEALMNKFGFQWPERLRCEHFPRHGAEQICVGQNHSEDGTPALLTTAPPSGLQPGLGERDCAAPCEPARPDGSMFFSHHHTRFARLWILTWSVLCCASTFFTVTTSLVAMQRFRYPERPIIFLSGCYTMVSVAYIAGFVLQERVVCNERFSEDGYRTVGQGTKKEGCTILFMMLYFFSMASSIWWVILSLTWFLAAGMKWGHAAIEANSQYFHLAAWAVPAVKTITILAMGQIDGDLLSGVCFVGLNRLDPLRGFVLAPLFVYLFIGTSFLLAGFVSLFRIRTIMKHDGTKTEPLERLMVRIGVFSVLYTVPATIVIACYFYEQAFREHWERSWVSQHCKSLAIPCPAHYTPRTSPDFTVYMIKYLMTLIVGITSGFWIWSGKTLHSWRKFYTRLTNSRHGETT

SEQ ID NO: 42 - protein, 536 aa, Drosophila
ILPTLIQGVQRYDQSPLDASPYYRSGGGLMASSGTELDGLPHHNRCEPITISICKNIPYNMTIMPNLIGHTKQEEAGLEVHQFAPLVKIGCSDDLQLFLCSLYVPVCTILERPIPPCRSLCESARVCEKLMKTYNFNWPENLECSKFPVHGGEDLCVAENTTSSASTAATPTRSVAVGGKDLHDCGAPCHAMFFPERERTVLRYWVGSWAAVCVASCLFTVLTFLIDSSRFRYPERAIVFLAVCYLVVGCAYVAGLGAGDSVSCREPFPPPVKLGRLQMMSTITQGHRQTTSCTVLFMALYFCCMAAFAWWSCLAFAWFLAAGLKWGHEAIENKSHLFHLVAWAVPALQTISVLALAKVEGDILSGVCFVGQLDTHSLGAFLILPLCIYLSIGALFLLAGFISLFRIRTVMKTDGKRTDKLERLMLRIGFFSGLFILPAVGLLGCLFYEYYNFDEWMIQWHRDICKPFSIPCPAARAPGSPEARPIFQIFMVKYLCSMLVGVTSSVWLYSSKTMVSWRNFVERLQGKEPRTRAQAY

SEQ ID NO: 43 - protein, 570 aa, Drosophila
GLVLLLTSCRADGPLHSADHGMGGMGMGGHGLDASPAPGYGVPAIPKDPNLRCEEITIPMCRGIGYNMTSFPNEMNHETQDEAGLEVHQFWPLVEIKCSPDLKFFLCSMYTPICLEDYHKPLPVCRSVCERARSGCAPIMQQYSFEWPERMACEHLPLHGDPDNLCMEQPSYTEAGSGGSSGGSGGSGSGSGSGGKRKQGGSGSGGSGAGGSSGSTSTKPCRGRQRIAGVPNCGIPCKGPFFSNDEKDFAGLWIALWSGLCFCSTLMTLTTFIIDTERFKYPERPIVFLSACYFMVAVGYLSRNFLQNEEIACDGLLLRESSTGPHSCTLVFLLTYFFGMASSIWWVILTFTWFLAAGLKWGNEAITKHSQYFHLAAWLIPTVQSVAVLLLSAVDGDPILGICYVGNLNPDHLKTFVLAPLFVYLVIGTTFLMAGFVSLFRIRSVIKQQGGVGAGVKADKLEKLMIRIGIFSVLYTVPATIVIGCYLYEAAYFEDWIKALACPCAQVKGPGKKPLYSVLMLKYFMALAVGITSGVWIWSGKTLESWRRFWRRLLGAPDRTGANQALIKQR

SEQ ID NO: 44 - protein, 647 aa, Homo sapiens
MAEEEAPKKSRAAGGGASWELCAGALSARLAEEGSGDAGGRRRPPVDPRRLARQLLLLLWLLEAPLLLGVRAQAAGQGPGQGPGPGQQPPPPPQQQQSGQQYNGERGISVPDHGYCQPISIPLCTDIAYNQTIMPNLLGHTNQEDAGLEVHQFYPLVKVQCSAELKFFLCSMYAPVCTVLEQALPPCRSLCERARQGCEALMNKFGFQWPDTLKCEKFPVHGAGELCVGQNTSDKGTPTPSLLPEFWTSNPQHGGGGHRGGFPGGAGASERGKFSCPRALKVPSYLNYHFLGEKDCGAPCEPTKVYGLMYFGPEELRFSRTWIGIWSVLCCASTLFTVLTYLVDMRRFSYPERPIIFLSGCYTAVAVAYIAGFLLEDRVVCNDKFAEDGARTVAQGTKKEGCTILFMMLYFFSMASSIWWVILSLTWFLAAGMKWGHEAIEANSQYFHLAAWAVPAIKTITILALGQVDGDVLSGVCFVGLNNVDALRGFVLAPLFVYLFIGTSFLLAGFVSLFRIRTIMKHDGTKTEKLEKLMVRIGVFSVLYTVPATIVIACYFYEQAFRDQWERSWVAQSCKSYAIPCPHLQAGGGAPPHPPMSPDFTVFMIKYLMTLIVGITSGFWIWSGKTLNSWRKFYTRLTNSKQGETTV

SEQ ID NO: 45 - protein, 626 aa, Mouse
MAEEAAPSESRAAGRLSLELCAEALPGRREEVGHEDTASHRRPRADPRRWASGLLLLLWLLEAPLLLGVRAQAAGQVSGPGQQAPPPPQPQQSGQQYNGERGISIPDHGYCQPISIPLCTDMAYNQTIMPNLLGHTNQEDAGLEVHQFYPLVKVQCSAELKFFLCSMYAPVCTVLEQALPPCRSLCERARQGCEALMNKFGFQWPDTLKCEKFPVHGAGELCVGQNTSDKGTPTPSLLPEFWTSNGQHGGGGYRGGYPGGAGTVERGKFSCPRALRVPSYLNYHFLGEKDCGAPCEPTKVYGLMYFGPEELRFSRTWIGIWSVLCCASTLFTVLTYLVDMPRFSYPERPIISLSGCYTAVAVAYIAGFLLEDRVVCNDKFAEDGARTVAQGTNKEGCTILFMMLYFFSMASSIWWVILSLTWFLAAGMKWGHEAIEANSQYFHLAAWAVPAIKTITILALGQVDGDVLSGVCFLGLNNVDALRGFVLAPLFVYLFIGTSFLLAGFVSLFRIRTIMKHDGTKTEKLEKLMVRIGVFSVLYTVPATIVIACYFYEQAFRDQWERSWVAQSCKSYAIPCPHLQGGGGVPPHPPMSPDFTVFMIKYLMTLNSWRKFYTRLTNSKQGETTV

SEQ ID NO: 46 - protein, 565 aa, Homo sapiens
MRPRSALPRLLLPLLLLPAAGPAQFHGEKGISIPDHGFCQPISIPLCTDIAYNQTIMPNLLGHTNQEDAGLEVHQFYPLVKVQCSPELRFFLCSMYAPVCTVLEQAIPPCRSICERARQGCEALMNKFGFQWPERLRCEHFPRHGAEQICVGQNHSEDGAPALLTTAPPPGLQPGAGGTPGGPGGGGAPPRYATLEHPFHCPRVLKVPSYLSYKFLGERDCAAPCEPARPDGSMFFSQEETRFARLWILTWSVLCCASTFFTVTTYLVDMQRFRYPERPIIFLSGCYTMVSVAYIAGFVLQERVVCNERFSEDGYRTVVQGTKKEGCTILFMMLYFFSMASSIWWVILSLTWFLAAGMKWGHEAIEANSQYFHLAAWAVPAVKTITILAMGQIDGDLLSGVCFVGLNSLDPLRGFVLAPLFVYLFIGTSFLLAGFVSLFRIRTIMKHDGTKTEKLERLMVRIGVFSVLYTVPATIVIACYFYEQAFREHWERSWVSQHCKSLAIPCPAHYTPRMSPDFTVYMIKYLMTLIVGITSGFWIWSGKTLHSWRKFYTRLTNSRHGETTV

SEQ ID NO: 47 - protein, 666 aa, Homo sapiens
MAMTWIVFSLWPLTVFMGHIGGHSLFSCEPITLRMCQDLPYNTTFMPNLLNHYDQQTAALAMEPFHPMVNLDCSRDFRPFLCALYAPICMEYGRVTLPCRRLCQRAYSECSKLMEMFGVPWPEDMECSRFPDCDEPYPRLVDLNLAGEPTEGAPVAVQRDYGFWCPRELKIDPDLGYSFLHVRDCSPPCPNMYFRREELSFARYFIGLISIICLSATLFTFLTFLIDVTRFRYPERPIIFYAVCYMMVSLIFFIGFLLEDRVACNASIPAQYKASTVTQGSHNKACTMLFMILYFFTMAGSVWWVILTITWFLAAVPKWGSEAIEKKALLFHASAWGIPGTLTIILLAMNKIEGDNISGVCFVGLYDVDALRYFVLAPLCLYVVVGVSLLLAGIISLNRVRIEIPLEKENQDKLVKFMIRIGVFSILYLVPLLVVIGCYFYEQAYRGIWETTWIQERCREYHIPCPYQVTQMSRPDLILFLMKYLMALIVGIPSVFWVGSKKTCFEWASFFHGRRKKEIVNESRQVLQEPDFAQSLLRDPNTPIIRKSRGTSTQGTSTHASSTQLAMVDDQRSKAGSIHSKVSSYHGSLHRSRDGRYTPCSYRGMEERLPHGSMSRLTDHSRHSSSHRLNEQSRHSSIRDLSNNPMTHITHGTSMNRVIEEDGTSA

SEQ ID NO: 48 - protein, 666 aa, Mouse
MAVSWIVFDLWLLTVFLGQIGGHSLFSCEPITLRMCQDLPYNTTFMPNLLNHYDQQTAALAMEPFHPMVNLDCSRDFRPFLCALYAPICMEYGRVTLPCRRLCQRAYSECSKLMEMFGVPWPEDMECSRFPDCDEPYPRLVDLNLVGDPTEGAPVAVQRDYGFWCPRELKIDPDLGYSFLHVRDCSPPCPNMYFRREELSFARYFIGLISIICLSATLFTFLTFLIDVTRFRYPERPIIFYAVCYMMVSLIFFIGFLLEDRVACNASSPAQYKASTVTQGSHNKACTMLFMVLYFFTMAGSVWWVILTITWFLAAVPKWGSEAIEKKALLFHASAWGIPGTLTIILLAMNKIEGDNISGVCFVGLYDVDALRYFVLAPLCLYVVVGVSLLLAGIISLNRVRIEIPLEKENQDKLVKFMIRIGVFSILYLVPLLVVIGCYFYEQAYRGIWETTWIQERCREYHIPCPYQVTQMSRPDLILFLMKYLMALIVGIPSIFWVGSKKTCFEWASFFHGRRKKEIVNESRQVLQEPDFAQSLLRDPNTPIIRKSRGTSTQGTSTHASSTQLAMVDDQRSKAGSVHSKVSSYHGSLHRSRDGRYTPCSYRGMEERLPHGSMSRLTDHSRHSSSHRLNEQSRHSSIRDLSNNPMTHITHGTSMNRVIEEDGTSA

SEQ ID NO: 49 - protein, 537 aa, Homo sapiens
MAWRGAGPSVPGAPGGVGLSLGLLLQLLLLLGPARGFGDEEERRCDPIRISMCQNLGYNVTKMPNLVGHELQTDAELQLTTFTPLIQYGCSSQLQFFLCSVYVPMCTEKINIPIGPCGGMCLSVKRRCEPVLKEFGFAWPESLNCSKFPPQNDHNHMCMEGPGDEEVPLPHKTPIQPGEECHSVGTNSDQYIWVKRSLNCVLKCGYDAGLYSRSAKEFTDIWMAVWASLCFISTAFTVLTFLIDSSRFSYPERPIIFLSMCYNIYSIAYIVRLTVGRERISCDFEEAAEPVLIQEGLKNTGCAIIFLLMYFFGMASSIWWVILTLTWFLAAGLKWGHEAIEMHSSYFHIAAWAIPAVKTIVILIMRLVDADELTGLCYVGNQNLDALTGFVVAPLFTYLVIGTLFIAAGLVALFKIRSNLQKDGTKTDKLERLMVKIGVFSVLYTVPATCVIACYFYEISNWALFRYSADDSNMAVEMLKIFMSLLVGITSGMWIWSAKTLHTWQKCSNRLVNSGKVKREKRGNGWVKPGKGSETVV

SEQ ID NO: 50 - protein, 537 aa, Mouse
MAWPGTGPSSRGAPGGVGLRLGLLLQFLLLLRPTLGFGDEEERRCDPIRIAMCQNLGYNVTKMPNLVGHELQTDAELQLTTFTPLIQYGCSSQLQFFLCSVYVPMCTEKINIPIGPCGGMCLSVKRRCEPVLREFGFAWPDTLNCSKFPPQNDHNHMCMEGPGDEEVPLPHKTPIQPGEECHSVGSNSDQYIWVKRSLNCVLKCGYDAGLYSRSAKEFTDIWMAVWASLCFISTTFTVLTFLIDSSRFSYPERPIIFLSMCYNIYSIAYIVRLTVGRERISCDFEEAAEPVLIQEGLKNTGCAIIFLLMYFFGMASSIWWVILTLTWFLAAGLKWGHEAIEMHSSYFHIAAWAIPAVKTIVILIMRLVDADELTGLCYVGNQNLDALTGFVVAPLFTYLVIGTLFIAAGLVALFKIRSNLQKDGTKTDKLERLMVKIGVFSVLYTVPATCVIACYFYEISNWALFRYSADDSNMAVEMLKIFMSLLVGITSGMWIWSAKTLHTWQKCSNRLVNSGKVKREKRGNGWVKPGKGNETVV

SEQ ID NO: 51 - protein, 585 aa, Homo sapiens
MARPDPSAPPSLLLLLLAQLVGRAAAASKAPVCQEITVPMCRGIGYNLTHMPNQFNHDTQDEAGLEVHQFWPLVEIQCSPDLRFFLCTMYTPICLPDYHKPLPPCRSVCERAKAGCSPLMRQYGFAWPERMSCDRLPVLGRDAEVLCMDYNRSEATTAPPRPFPAKPTLPGPPGAPASGGECPAGGPFVCKCREPFVPILKESHPLYNKVRTGQVPNCAVPCYQPSFSADERTFATFWIGLWSVLCFISTSTTVATFLIDMDTFRYPERPIIFLSACYLCVSLGFLVRLVVGHASVACSREHNHIHYETTGPALCTIVFLLVYFFGMASSIWWVILSLTWFLAAAMKWGNEAIAGYGQYFHLAAWLIPSVKSITALALSSVDGDPVAGICYVGNQNLNSLRRFVLGPLVLYLLVGTLFLLAGFVSLFRIRSVIKQGGTKTDKLEKLMIRIGIFTLLYTVPASIVVACYLYEQHYRESWEAALTCACPGHDTGQPRAKPEYWVLMLKYFMCLVVGITSGVWIWSGKTVESWRRFTSRCCCRPRRGHKSGGAMAAGDYPEASAALTGRTGPPGPAATYHKQVSLSHV

SEQ ID NO: 52 - protein, 706 aa, Homo sapiens
MEMFTFLLTCIFLPLLRGHSLFTCEPITVPRCMKMAYNMTFFPNLMGHYDQSIAAVEMEHFLPLANLECSPNIETFLCKAFVPTCIEQIHVVPPCRKLCEKVYSDCKKLIDTFGIRWPEELECDRLQYCDETVPVTFDPHTEFLGPQKKTEQVQRDIGFWCPRHLKTSGGQGYKFLGIDQCAPPCPNMYFKSDELEFAKSFIGTVSIFCLCATLFTFLTFLIDVRRFRYPERPIIYYSVCYSIVSLMYFIGFLLGDSTACNKADEKLELGDTVVLGSQNKACTVLFMLLYFFTMAGTVWWVILTITWFLAAGRKWSCEAIEQKAVWFHAVAWGTPGFLTVMLLAMNKVEGDNISGVCFVGLYDLDASRYFVLLPLCLCVFVGLSLLLAGIISLNHVRQVIQHDGRNQEKLKKFMIRIGVFSGLYLVPLVTLLGCYVYEQVNRITWEITWVSDHCRQYHIPCPYQAKAKARPELALFMIKYLMTLIVGISAVFWVGSKKTCTEWAGFFKRNRKRDPISESRRVLQESCEFFLKHNSKVKHKKKHYKPSSHKLKVISKSMGTSTGATANHGTSAVAITSHDYLGQETLTEIQTSPETSMREVKADGASTPRLREQDCGEPASPAASISRLSGEQVDGKGQAGSVSESARSEGRISPKSDITDTGLAQSNNLQVPSSSEPSSLKGSTSLLVHPVSGVRKEQGGGCHSDT

SEQ ID NO: 53 - protein, 709 aa, Mouse
MERSPFLLACILLPLVRGHSLFTCEPITVPRCMKMTYNMTFFPNLMGHYDQGIAAVEMGHFLHLANLECSPNIEMFLCQAFIPTCTEQIHVVLPCRKLCEKIVSDCKKLMDTFGIRWPEELECNRLPHCDDTVPVTSHPHTELSGPQKKSDQVPRDIGFWCPKHLRTSGDQGYRFLGIEQCAPPCPNMYFKSDELDFAKSFIGIVSIFCLCATLFTFLTFLIDVRRFRYPERPIIYYSVCYSIVSLMYFVGFLLGNSTACNKADEKLELGDTVVLGSKNKACSVVFMFLYFFTMAGTVWWVILTITWFLAAGRKWSCEAIEQKAVWFHAVAWGAPGFLTVMLLAMNKVEGDNISGVCFVGLYDLDASRYFVLLPLCLCVFVGLSLLLAGIISLNHVRQVIQHDGRNQEKLKKFMIRIGVFSGLYLVPLVTLLGCYVYELVNRITWEMTWFSDHCHQYRIPCPYQANPKARPELALFMIKYLMTLIVGISAVFWVGSKKTCTEWAGFFKRNRKRDPISESRRVLQESCEFFLKHNSKVKHKKKHGAPGPHRLKVISKSMGTSTGATTNHGTSAMAIADHDYLGQETSTEVHTSPEASVKEGRADRANTPSAKDRDCGESAGPSSKLSGNRNGRESRAGGLKERSNGSEGAPSEGRVSPKSSVPETGLIDCSTSQAASSPEPTSLKGSTSLPVHSASRARKEQGAGSHSDA

SEQ ID NO: 54 - protein, 574 aa, Homo sapiens
MRDPGAAAPLSSLGLCALVLALLGALSAGAGAQPYHGEKGISVPDHGFCQPISIPLCTDIAYNQTILPNLLGHTNQEDAGLEVHQFYPLVKVQCSPELRFFLCSMYAPVCTVLDQAIPPCRSLCERARQGCEALMNKFGFQWPERLRCENFPVHGAGEICVGQNTSDGSGGPGGGPTAYPTAPYLPDLPFTALPPGASDGRGRPAFPFSCPRQLKVPPYLGYRFLGERDCGAPCEPGRANGLMYFKEEERRFARLWVGVWSVLCCASTLFTVLTYLVDMRRFSYPERPIIFLSGCYFMVAVAHVAGFLLEDRAVCVERFSDDGYRTVAQGTKKEGCTILFMVLYFFGMASSIWWVILSLTWFLAAGMKWGHEAIEANSQYFHLAAWAVPAVKTITILAMGQVDGDLLSGVCYVGLSSVDALRGFVLAPLFVYLFIGTSFLLAGFVSLFRIRTIMKHDGTKTEKLEKLMVRIGVFSVLYTVPATIVLACYFYEQAFREHWERTWLLQTCKSYAVPCPPGHFPPMSPDFTVFMIKYLMTMIVGITTGFWIWSGKTLQSWRRFYHRLSHSSKGETAV

SEQ ID NO: 55 - protein, 572 aa, Mouse
MRGPGTAASHSPLGLCALVLALLGALPTDTRAQPYHGEKGISVPDHGFCQPISIPLCTDIAYNQTILPNLLGHTNQEDAGLEVHQFYPLVKVQCSPELRFFLCSMYAPVCTVLDQAIPPCRSLCERARQGCEALMNKFGFQWPERLRCENFPVHGAGEICVGQNTSDGSGGAGGSPTAYPTAPYLPDPPFTAMSPSDGRGRLSFPFSCPRQLKVPPYLGYRFLGERDCGAPCEPGRANGLMYFKEEERRFARLWVGVWSVLSCASTLFTVLTYLVDMRRFSYPERPIIFLSGCYFMVAVAHVAGFLLEDRAVCVERFSDDGYRTVAQGTKKEGCTILFMVLYFFGMASSIWWVILSLTWFLAAGMKWGHEAIEANSQYFHLAAWAVPAVKTITILAMGQVDGDLLSGVCYVGLSSVDALRGFVLAPLFVYLFIGTSFLLAGFVSLFRIRTIMKHDGTKTEKLEKLMVRIGVFSVLYTVPATIVLACYFYEQAFREHWERTWLLQTCKSYAVPCPPRHFSPMSPDFTVFMIKYLMTMIVGITTGFWIWSGKTLQSWRRFYHRLSHSSKGETAV

SEQ ID NO: 56 - protein, 694 aa, Homo sapiens
MEWGYLLEVTSLLAALALLQRSSGAAAASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYNRTDLTTAAPSPPRRLPPPPPGEQPPSGSGHGRPPGARPPHRGGGRGGGGGDAAAPPARGGGGGGKARPPGGGAAPCEPGCQCRAPMVSVSSERHPLYNRVKTGQIANCALPCHNPFFSQDERAFTVFWIGLWSVLCFVSTFATVSTFLIDMERFKYPERPIIFLSACYLFVSVGYLVRLVAGHEKVACSGGAPGAGGAGGAGGAAAGAGAAGAGAGGPGGRGEYEELGAVEQHVRYETTGPALCTVVFLLVYFFGMASSIWWVILSLTWFLAAGMKWGNEAIAGYSQYFHLAAWLVPSVKSIAVLALSSVDGDPVAGICYVGNQSLDNLRGFVLAPLVIYLFIGTMFLLAGFVSLFRIRSVIKQQDGPTKTHKLEKLMIRLGLFTVLYTVPAAVVVACLFYEQHNRPRWEATHNCPCLRDLQPDQARRPDYAVFMLKYFMCLVVGITSGVWVWSGKTLESWRSLCTRCCWASKGAAVGGGAGATAAGGGGGPGGGGGGGPGGGGGPGGGGGSLYSDVSTGLTWRSGTASSVSYPKQMPLSQV

SEQ ID NO: 57 - protein, 685 aa, Mouse
MEWGYLLEVTSLLAALAVLQRSSGAAAASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYNRTDLTTAAPSPPRRLPPPPPPGEQPPSGSGHSRPPGARPPHRGGSSRGSGDAAAAPPSRGGKARPPGGGAAPCEPGCQCRAPMVSVSSERHPLYNRVKTGQIANCALPCHNPFFSQDERAFTVFWIGLWSVLCFVSTFATVSTFLIDMERFKYPERPIIFLSACYLFVSVGYLVRLVAGHEKVACSGGAPGAGGRGGAGGAAAAGAGAAGRGASSPGARGEYEELGAVEQHVRYETTGPALCTVVFLLVYFFGMASSIWWVILSLTWFLAAGMKWGNEAIAGYSQYFHLAAWLVPSVKSIAVLALSSVDGDPVAGICYVGNQSLDNLRGFVLAPLVIYLFIGTMFLLAGFVSLFRIRSVIKQQGGPTKTHKLEKLMIRLGLFTVLYTVPAAVVVACLFYEQHNRPRWEATHNCPCLRDLQPDQARRPDYAVFMLKYFMCLVVGITSGVWVWSGKTLESWRALCTRCCWASKGAAVGAGAGGSGPGGSGPGPGGGGGHGGGGGSLYSDVSTGLTWRSGTASSVSYPKQMPLSQV

SEQ ID NO: 58 - protein, 591 aa, Homo sapiens
MAVAPLRGALLLWQLLAAGGAALEIGRFDPERGRGAAPCQAVEIPMCRGIGYNLTRMPNLLGHTSQGEAAAELAEFAPLVQYGCHSHLRFFLCSLYAPMCTDQVSTPIPACRPMCEQARLRCAPIMEQFNFGWPDSLDCARLPTRNDPHALCMEAPENATAGPAEPHKGLGMLPVAPRPARPPGDLGPGAGGSGTCENPEKFQYVEKSRSCAPRCGPGVEVFWSRRDKDFALVWMAVWSALCFFSTAFTVLTFLLEPHRFQYPERPIIFLSMCYNVYSLAFLIRAVAGAQSVACDQEAGALYVIQEGLENTGCTLVFLLLYYFGMASSLWWVVLTLTWFLAAGKKWGHEAIEAHGSYFHMAAWGLPALKTIVILTLRKVAGDELTGLCYVASTDAAALTGFVLVPLSGYLVLGSSFLLTGFVALFHIRKIMKTGGTNTEKLEKLMVKIGVFSILYTVPATCVIVCYVYERLNMDFWRLRATEQPCAAAAGPGGRRDCSLPGGSVPTVAVFMLKIFMSLVVGITSGVWVWSSKTFQTWQSLCYRKIAAGRARAKACRAPGSYGRGTHCHYKAPTVVLHMTKTDPSLENPTHL

SEQ ID NO: 59 - protein, 591 aa, Mouse
MAVPPLLRGALLLWQLLATGGAALEIGRFDPERGRGPAPCQAMEIPMCRGIGYNLTRMPNLLGHTSQGEAAAQLAEFSPLVQYGCHSHLRFFLCSLYAPMCTDQVSTPIPACRPMCEQARLRCAPIMEQFNFGWPDSLDCARLPTRNDPHALCMEAPENTAGPTEPHKGLGMLPVAPRPARPPGDSAPGPGSGGTCDNPEKFQYVEKSRSCAPRCGPGVEVFWSRRDKDFALVWMAVWSALCFFSTAFTVFTFLLEPHRFQYPERPIIFLSMCYNVYSLAFLIRAVAGAQSVACDQEAGALYVIQEGLENTGCTLVFLLLYYFGMASSLWWVVLTLTWFLAAGKKWGHEAIEAHGSYFHMAAWGLPALKTIVVLTLRKVAGDELTGLCYVASMDPAALTGFVLVPLSCYLVLGTSFLLTGFVALFHIRKIMKTGGTNTEKLEKLMVKIGVFSILYTVPATCVIVCYVYERLNMDFWRLRATEQPCTAATVPGGRRDCSLPGGSVPTVAVFMLKIFMSLVVGITSGVWVWSSKTFQTWQSLCYRKMAAGRARAKACRTPGGYGRGTHCHYKAPTVVLHMTKTDPSLENPTHL

SEQ ID NO: 60 - protein, 581 aa, Homo sapiens
  Variant (464): Xaa = any amino acid
MQRPGPRLWLVLQVMGSCAAISSMDMERPGDGKCQPIEIPMCKDIGYNMTRMPNLMGHENQREAAIQLHEFAPLVEYGCHGHLRFFLCSLYAPMCTEQVSTPIPACRVMCEQARLKCSPIMEQFNFKWPDSLDCRKLPNKNDPNYLCMEAPNNGSDEPTRGSGLFPPLFRPQRPHSAQEHPLKDGGPGRGGCDNPGKFHHVEKSASCAPLCTPGVDVYWSREDKRFAVVWLAIWAVLCFFSSAFTVLTFLIDPARFRYPERPIIFLSMCYCVYSVGYLIRLFAGAESIACDRDSGQLYVIQEGLESTGCTLVFLVLYYFGMASSLWWVVLTLTWFLAAGKKWGHEAIEANSSYFHLAAWAIPAVKTILILVMRRVAGDELTGVCYVGSMDVNALTGFVLIPLACYLVIGTSFILSGFVALFHIRRVMKTGGENTDKLEKLMVRIGLFSVLYTVPATCVIACYFXEHLNMDYWKILAAQHKCKMNNQTKTLDCLMAASIPAVEIFMVKIFMLLVVGITSGMWIWTSKTLQSWQQVCSRRLKKKSRRKPASVITSGGIYKKAQHPQKTHHGKYEIPAQSPTCV

SEQ ID NO: 61 - protein, 319 aa, Homo sapiens
MAEEEAPKKSRAAGGGASWELCAGALSARLAEEGSGDAGGRRRPPVDPRRLARQLLLLLWLLEAPLLLGVRAQAAGQGPGQGPGPGQQPPPPPQQQQSGQQYNGERGISVPDHGYCQPISIPLCTDIAYNQTIMPNLLGHTNQEDAGLEVHQFYPLVKVQCSAELKFFLCSMYAPVCTVLEQALPPCRSLCERARQGCEALMNKFGFQWPDTLKCEKFPVHGAGELCVGQNTSDKGTPTPSLLPEFWTSNPQHGGGGHRGGFPGGAGASERGKFSCPRALKVPSYLNYHFLGEKDCGAPCEPTKVYGLMYFGPEELRFS

SEQ ID NO: 62 - protein, 314 aa, Mouse
MAEEAAPSESRAAGRLSLELCAEALPGRREEVGHEDTASHRRPRADPRRWASGLLLLLWLLEAPLLLGVRAQAAGQVSGPGQQAPPPPQPQQSGQQYNGERGISIPDHGYCQPISIPLCTDMAYNQTIMPNLLGHTNQEDAGLEVHQFYPLVKVQCSAELKFFLCSMYAPVCTVLEQALPPCRSLCERARQGCEALMNKFGFQWPDTLKCEKFPVHGAGELCVGQNTSDKGTPTPSLLPEFWTSNGQHGGGGYRGGYPGGAGTVERGKFSCPRALRVPSYLNYHFLGEKDCGAPCEPTKVYGLMYFGPEELRFS

SEQ ID NO: 63 - protein, 244 aa, Homo sapiens
MRPRSALPRLLLPLLLLPAAGPAQFHGEKGISIPDHGFCQPISIPLCTDIAYNQTIMPNLLGHTNQEDAGLEVHQFYPLVKVQCSPELRFFLCSMYAPVCTVLEQAIPPCRSICERARQGCEALMNKFGFQWPERLRCEHFPRHGAEQICVGQNHSEDGAPALLTTAPPPGLQPGAGGTPGGPGGGGAPPRYATLEHPFHCPRVLKVPSYLSYKFLGERDCAAPCEPARPDGSMFFSQEETRFA

SEQ ID NO: 64 - protein, 202 aa, Homo sapiens
MAMTWIVFSLWPLTVFMGHIGGHSLFSCEPITLRMCQDLPYNTTFMPNLLNHYDQQTAALAMEPFHPMVNLDCSRDFRPFLCALYAPICMEYGRVTLPCRRLCQRAYSECSKLMEMFGVPWPEDMECSRFPDCDEPYPRLVDLNLAGEPTEGAPVAVQRDYGFWCPRELKIDPDLGYSFLHVRDCSPPCPNMYFRREELSFA

SEQ ID NO: 65 - protein, 202 aa, Mouse
MAVSWIVFDLWLLTVFLGQIGGHSLFSCEPITLRMCQDLPYNTTFMPNLLNHYDQQTAALAMEPFHPMVNLDCSRDFRPFLCALYAPICMEYGRVTLPCRRLCQRAYSECSKLMEMFGVPWPEDMECSRFPDCDEPYPRLVDLNLVGDPTEGAPVAVQRDYGFWCPRELKIDPDLGYSFLHVRDCSPPCPNMYFRREELSFA

SEQ ID NO: 66 - protein, 219 aa, Homo sapiens
MAWRGAGPSVPGAPGGVGLSLGLLLQLLLLLGPARGFGDEEERRCDPIRISMCQNLGYNVTKMPNLVGHELQTDAELQLTTFTPLIQYGCSSQLQFFLCSVYVPMCTEKINIPIGPCGGMCLSVKRRCEPVLKEFGFAWPESLNCSKFPPQNDHNHMCMEGPGDEEVPLPHKTPIQPGEECHSVGTNSDQYIWVKRSLNCVLKCGYDAGLYSRSAKEFT

SEQ ID NO: 67 - protein, 219 aa, Mouse
MAWPGTGPSSRGAPGGVGLRLGLLLQFLLLLRPTLGFGDEEERRCDPIRIAMCQNLGYNVTKMPNLVGHELQTDAELQLTTFTPLIQYGCSSQLQFFLCSVYVPMCTEKINIPIGPCGGMCLSVKRRCEPVLREFGFAWPDTLNCSKFPPQNDHNHMCMEGPGDEEVPLPHKTPIQPGEECHSVGSNSDQYIWVKRSLNCVLKCGYDAGLYSRSAKEFT

SEQ ID NO: 68 - protein, 235 aa, Homo sapiens
MARPDPSAPPSLLLLLLAQLVGRAAAASKAPVCQEITVPMCRGIGYNLTHMPNQFNHDTQDEAGLEVHQFWPLVEIQCSPDLRFFLCTMYTPICLPDYHKPLPPCRSVCERAKAGCSPLMRQYGFAWPERMSCDRLPVLGRDAEVLCMDYNRSEATTAPPRPFPAKPTLPGPPGAPASGGECPAGGPFVCKCREPFVPILKESHPLYNKVRTGQVPNCAVPCYQPSFSADERTFA

SEQ ID NO: 69 - protein, 198 aa, Homo sapiens
MEMFTFLLTCIFLPLLRGHSLFTCEPITVPRCMKMAYNMTFFPNLMGHYDQSIAAVEMEHFLPLANLECSPNIETFLCKAFVPTCIEQIHVVPPCRKLCEKVYSDCKKLIDTFGIRWPEELECDRLQYCDETVPVTFDPHTEFLGPQKKTEQVQRDIGFWCPRHLKTSGGQGYKFLGIDQCAPPCPNMYFKSDELEFA

SEQ ID NO: 70 - protein, 198 aa, Mouse
MERSPFLLACILLPLVRGHSLFTCEPITVPRCMKMTYNMTFFPNLMGHYDQGIAAVEMGHFLHLANLECSPNIEMFLCQAFIPTCTEQIHVVLPCRKLCEKIVSDCKKLMDTFGIRWPEELECNRLPHCDDTVPVTSHPHTELSGPQKKSDQVPRDIGFWCPKHLRTSGDQGYRFLGIEQCAPPCPNMYFKSDELDFA

SEQ ID NO: 71 - protein, 253 aa, Homo sapiens
MRDPGAAAPLSSLGLCALVLALLGALSAGAGAQPYHGEKGISVPDHGFCQPISIPLCTDIAYNQTILPNLLGHTNQEDAGLEVHQFYPLVKVQCSPELRFFLCSMYAPVCTVLDQAIPPCRSLCERARQGCEALMNKFGFQWPERLRCENFPVHGAGEICVGQNTSDGSGGPGGGPTAYPTAPYLPDLPFTALPPGASDGRGRPAFPFSCPRQLKVPPYLGYRFLGERDCGAPCEPGRANGLMYFKEEERRFA

SEQ ID NO: 72 - protein, 251 aa, Mouse
MRGPGTAASHSPLGLCALVLALLGALPTDTRAQPYHGEKGISVPDHGFCQPISIPLCTDIAYNQTILPNLLGHTNQEDAGLEVHQFYPLVKVQCSPELRFFLCSMYAPVCTVLDQAIPPCRSLCERARQGCEALMNKFGFQWPERLRCENFPVHGAGEICVGQNTSDGSGGAGGSPTAYPTAPYLPDPPFTAMSPSDGRGRLSFPFSCPRQLKVPPYLGYRFLGERDCGAPCEPGRANGLMYFKEEERRFA

SEQ ID NO: 73 - protein, 277 aa, Homo sapiens
MEWGYLLEVTSLLAALALLQRSSGAAAASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYNRTDLTTAAPSPPRRLPPPPPGEQPPSGSGHGRPPGARPPHRGGGRGGGGGDAAAPPARGGGGGGKARPPGGGAAPCEPGCQCRAPMVSVSSERHPLYNRVKTGQIANCALPCHNPFFSQDERAFT

SEQ ID NO: 74 - protein, 274 aa, Mouse
MEWGYLLEVTSLLAALAVLQRSSGAAAASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYNRTDLTTAAPSPPRRLPPPPPPGEQPPSGSGHSRPPGARPPHRGGSSRGSGDAAAAPPSRGGKARPPGGGAAPCEPGCQCRAPMVSVSSERHPLYNRVKTGQIANCALPCHNPFFSQDERAFT

SEQ ID NO: 75 - protein, 231 aa, Homo sapiens
MAVAPLRGALLLWQLLAAGGAALEIGRFDPERGRGAAPCQAVEIPMCRGIGYNLTRMPNLLGHTSQGEAAAELAEFAPLVQYGCHSHLRFFLCSLYAPMCTDQVSTPIPACRPMCEQARLRCAPIMEQFNFGWPDSLDCARLPTRNDPHALCMEAPENATAGPAEPHKGLGMLPVAPRPARPPGDLGPGAGGSGTCENPEKFQYVEKSRSCAPRCGPGVEVFWSRRDKDFA

SEQ ID NO: 76 - protein, 232 aa, Mouse
MAVPPLLRGALLLWQLLATGGAALEIGRFDPERGRGPAPCQAMEIPMCRGIGYNLTRMPNLLGHTSQGEAAAQLAEFSPLVQYGCHSHLRFFLCSLYAPMCTDQVSTPIPACRPMCEQARLRCAPIMEQFNFGWPDSLDCARLPTRNDPHALCMEAPENATAGPTEPHKGLGMLPVAPRPARPPGDSAPGPGSGGTCDNPEKFQYVEKSRSCAPRCGPGVEVFWSRRDKDFA

SEQ ID NO: 77 - protein, 227 aa, Homo sapiens
MQRPGPRLWLVLQVMGSCAAISSMDMERPGDGKCQPIEIPMCKDIGYNMTRMPNLMGHENQREAAIQLHEFAPLVEYGCHGHLRFFLCSLYAPMCTEQVSTPIPACRVMCEQARLKCSPIMEQFNFKWPDSLDCRKLPNKNDPNYLCMEAPNNGSDEPTRGSGLFPPLFRPQRPHSAQEHPLKDGGPGRGGCDNPGKFHHVEKSASCAPLCTPGVDVYWSREDKRFA

SEQ ID NO: 78 - protein, 29 aa, Homo sapiens
DRVVCNDKFAEDGARTVAQGTKKEGCTIL

SEQ ID NO: 79 - protein, 29 aa, Mouse
DRVVCNDKFAEDGARTVAQGTNKEGCTIL

SEQ ID NO: 80 - protein, 29 aa, Homo sapiens
ERVVCNERFSEDGYRTVVQGTKKEGCTIL

SEQ ID NO: 81 - protein, 30 aa, Homo sapiens
DRVACNASIPAQYKASTVTQGSHNKACTML

SEQ ID NO: 82 - protein, 30 aa, Mouse
DRVACNASSPAQYKASTVTQGSHNKACTML

SEQ ID NO: 83 - protein, 29 aa, Homo sapiens
RERISCDFEEAAEPVLIQEGLKNTGCAII

SEQ ID NO: 84 - protein, 29 aa, Mouse
RERISCDFEEAAEPVLIQEGLKNTGCAII

SEQ ID NO: 85 - protein, 26 aa, Homo sapiens
HASVACSREHNHIHYETTGPALCTIV

SEQ ID NO: 86 - protein, 30 aa, Homo sapiens
DSTACNKADEKLELGDTVVLGSQNKACTVL

SEQ ID NO: 87 - protein, 30 aa, Mouse
NSTACNKADEKLELGDTVVLGSKNKACSVV

SEQ ID NO: 88 - protein, 29 aa, Homo sapiens
DRAVCVERFSDDGYRTVAQGTKKEGCTIL

SEQ ID NO: 89 - protein, 29 aa, Mouse
DRAVCVERFSDDGYRTVAQGTKKEGCTIL

SEQ ID NO: 90 - protein, 65 aa, Homo sapiens
HEKVACSGGAPGAGGAGGAGGAAAGAGAAGAGAGGPGGRGEYEELGAVEQHVRYETTGPALCTVV

SEQ ID NO: 91 - protein, 66 aa, Mouse
HEKVACSGGAPGAGGRGGAGGAAAAGAGAAGRGASSPGARGEYEELGAVEQHVRYETTGPALCTVV

SEQ ID NO: 92 - protein, 28 aa, Homo sapiens
AQSVACDQEAGALYVIQEGLENTGCTLV

SEQ ID NO: 93 - protein, 28 aa, Mouse
AQSVACDQEAGALYVIQEGLENTGCTLV

SEQ ID NO: 94 - protein, 28 aa, Homo sapiens
AESIACDRDSGQLYVIQEGLESTGCTLV

SEQ ID NO: 95 - protein, 25 aa, Homo sapiens
GQVDGDVLSGVCFVGLNNVDALRGF

SEQ ID NO: 96 - protein, 25 aa, Mouse
GQVDGDVLSGVCFLGLNNVDALRGF

SEQ ID NO: 97 - protein, 25 aa, Homo sapiens
GQIDGDLLSGVCFVGLNSLDPLRGF

SEQ ID NO: 98 - protein, 25 aa, Homo sapiens
NKIEGDNISGVCFVGLYDVDALRYF

SEQ ID NO: 99 - protein, 25 aa, Mouse
NKIEGDNISGVCFVGLYDVDALRYF

SEQ ID NO: 100 - protein, 25 aa, Homo sapiens
RLVDADELTGLCYVGNQNLDALTGF

SEQ ID NO: 101 - protein, 25 aa, Mouse
RLVDADELTGLCYVGNQNLDALTGF

SEQ ID NO: 102 - protein, 25 aa, Homo sapiens
SSVDGDPVAGICYVGNQNLNSLRRF

SEQ ID NO: 103 - protein, 25 aa, Homo sapiens
NKVEGDNISGVCFVGLYDLDASRYF

SEQ ID NO: 104 - protein, 25 aa, Mouse
NKVEGDNISGVCFVGLYDLDASRYF

SEQ ID NO: 105 - protein, 25 aa, Homo sapiens
GQVDGDLLSGVCYVGLSSVDALRGF

SEQ ID NO: 106 - protein, 25 aa, Mouse
GQVDGDLLSGVCYVGLSSVDALRGF

SEQ ID NO: 107 - protein, 25 aa, Homo sapiens
SSVDGDPVAGICYVGNQSLDNLRGF

SEQ ID NO: 108 - protein, 25 aa, Mouse
SSVDGDPVAGICYVGNQSLDNLRGF

SEQ ID NO: 109 - protein, 25 aa, Homo sapiens
RKVAGDELTGLCYVASTDAAALTGF

SEQ ID NO: 110 - protein, 25 aa, Mouse
RKVAGDELTGLCYVASMDPAALTGF

SEQ ID NO: 111 - protein, 24 aa, Homo sapiens
RRVAGDELTGVCYVGSMDVNALTG

SEQ ID NO: 112 - protein, 39 aa, Homo sapiens
AFRDQWERSWVAQSCKSYAIPCPHLQAGGGAPPHPPMSP

SEQ ID NO: 113 - protein, 39 aa, Mouse
AFRDQWERSWVAQSCKSYAIPCPHLQGGGGVPPHPPMSP

SEQ ID NO: 114 - protein, 32 aa, Homo sapiens
AFREHWERSWVSQHCKSLAIPCPAHYTPRMSP

SEQ ID NO: 115 - protein, 32 aa, Homo sapiens
AYRGIWETTWIQERCREYHIPCPYQVTQMSRP

SEQ ID NO: 116 - protein, 32 aa, Mouse; identical to SEQ ID NO: 115

SEQ ID NO: 117 - protein, 17 aa, Homo sapiens
SNWALFRYSADDSNMAV

SEQ ID NO: 118 - protein, 17 aa, Mouse
SNWALFRYSADDSNMAV

SEQ ID NO: 119 - protein, 26 aa, Homo sapiens
HYRESWEAALTCACPGHDTGQPRAKP

SEQ ID NO: 120 - protein, 32 aa, Homo sapiens
VNRITWEITWVSDHCRQYHIPCPYQAKAKARP

SEQ ID NO: 121 - protein, 32 aa, Mouse
VNRITWEMTWFSDHCHQYRIPCPYQANPKARP

SEQ ID NO: 122 - protein, 32 aa, Homo sapiens
AFREHWERTWLLQTCKSYAVPCPPGHFPPMSP

SEQ ID NO: 123 - protein, 32 aa, Mouse
AFREHWERTWLLQTCKSYAVPCPPRHFSPMSP

SEQ ID NO: 124 - protein, 26 aa, Homo sapiens
HNRPRWEATHNCPCLRDLQPDQARRP

SEQ ID NO: 125 - protein, 26 aa, Mouse
HNRPRWEATHNCPCLRDLQPDQARRP

SEQ ID NO: 126 - protein, 35 aa, Homo sapiens
LNMDFWRLRATEQPCAAAAGPGGRRDCSLPGGSVP

SEQ ID NO: 127 - protein, 35 aa, Mouse
LNMDFWRLRATEQPCTAATVPGGRRDCSLPGGSVP

SEQ ID NO: 128 - protein, 33 aa, Homo sapiens
LNMDYWKILAAQHKCKMNNQTKTLDCLMAASIP

SEQ ID NO: 129 - protein, 48 aa, Homo sapiens
VGQNTSDKGTPSLLPEFWTSNPQHGGGHRGGFPGGAGASERGKFSCPR

SEQ ID NO: 130 - protein, 51 aa, Homo sapiens
VGQNHSEDGAPALLTTAPPPGLQPGAGGTPGGPGGGGAPPRYATLEHPFHC

SEQ ID NO: 131 - protein, 26 aa, Homo sapiens
LVDLNLAGEPTEGAPVAVQRDYGFWC

SEQ ID NO: 132 - protein, 20 aa, Homo sapiens
CMEGPGDEEVPLPHKTPIQP

SEQ ID NO: 133 - protein, 46 aa, Homo sapiens
CMDYNRSEATTAPPRPFPAKPTLPGPPGAPASGGECPAGGPFVCKC

SEQ ID NO: 134 - protein, 26 aa, Homo sapiens
TFDPHTEFLGPQKKTEQVQRDIGFMC

SEQ ID NO: 135 - protein, 50 aa, Homo sapiens
VGQNTSDGSGGPGGGPTAYPTAPYLPDLPFTALPPGASDGRGRPAFPFSC

SEQ ID NO: 136 - protein, 86 aa, Homo sapiens
CMDYNRTDLTTAAPSPPRRLPPPPPGEQPPSGSGHGRPPGARPPHRGGGRGGGGDAAAPPARGGGGGGKARPPGGGAAPCEPGCQC

SEQ ID NO: 137 - protein, 37 aa, Homo sapiens
CMEAPENATAGPAEPHKGLGMLPVAPRPARPPGDLGP

SEQ ID NO: 138 - protein, 38 aa, Homo sapiens
NYLCVEAPNNGSDEPTRGSGLFPPLFRPQRPHSAQEHP

(19) United States
(12) Patent Application Publication
(10) Pub. No.: US 2003/0044409 A1
(43) Pub. Date: Mar. 6, 2003

(54) Immunologic compositions and methods for studying and treating cancers expressing frizzled antigens

(76) Inventors: Dennis A. Carson, Del Mar, CA (US); Maripat Corr, San Diego, CA (US); Chae-Seo Rhee, San Diego, CA (US); Lorenzo M. Leoni, San Diego, CA (US); Malini Sen, San Diego, CA (US)

Correspondence Address: MORRISON & FOERSTER LLP, 3811 VALLEY CENTRE DRIVE, SUITE 500, SAN DIEGO, CA, 92130-2332, US

(21) Appl. No.: 09/847,102

(22) Filed: May 1, 2001

(51) Int. Cl. C07H 21/04; C07K 16/30; A61K 39/395; C12P 21/02; C12N 5/06; C07K 14/705

(52) U.S. Cl. 424/143.1; 530/388.22; 435/69.1; 435/320.1; 435/334; 536/23.53; 435/325; 530/350

(57) ABSTRACT

This invention is in the field of immunology. More specifically, it relates to compositions and methods for identifying, treating and preventing cancer by targeting the extracellular domains of the frizzled receptor family of proteins.

### TECHNICAL FIELD

This invention is in the field of immunology. More specifically, it relates to compositions and methods for identifying, treating and preventing cancer by targeting the extracellular domains of the frizzled receptor family of proteins.

### BACKGROUND OF THE INVENTION

Many adult cancers arise from small populations of residual tissue stem cells that have a high rate of cell proliferation. These tissue stem cells express various different cell surface receptors and ligands that are used to direct tissue pattern formation and cellular differentiation during development of the embryo, but since these receptors and ligands are not needed in adults, their expression is often very low in differentiated cells. Thus, targeting the immunological differences between the receptors that are expressed by cancers arising from residual tissue stem cells and those found on normal cells may provide for useful cancer therapies.

In order for cell surface receptors and their associated ligands to be suitable targets for immunotherapies, they should have certain preferred characteristics. First, they should be expressed on the surface of the malignant cells, and to a much lesser degree on normal cells. Second, they should have areas of secondary structures that give rise to conformations which are capable of being recognized by antibodies, cytotoxic T cells and/or drugs. Third, these areas of recognition should be sufficiently different from other cell surface receptors to avoid potentially damaging immunologic cross-reactions.

The G-protein coupled receptors (GPCRs) are particularly attractive targets for both passive and active immunotherapy, because many of these receptors have all three of these characteristics. In general, they contain seven membrane-spanning regions and a relatively short amino-terminal tail that is exposed into the extracellular environment. This “tail” often assumes a defined secondary structure which is unique to each receptor. In addition to the tail portion, there are other regions in-between the membrane-spanning regions that are also exposed on the cell surface. Accordingly, members of this gene family may be attractive targets for active and passive immunotherapies.

Frizzled antigens are a family of GPCR-like receptors that have binding sites for Wnt protein ligands, which are secreted molecules that act as upregulators of gene expression via the β-catenin cytoplasmic intermediate pathway. This receptor-ligand pair plays a role in embryonic development, and may play a role in cellular proliferation and the ultimate fate of cells during embryogenesis.

The presence of frizzled gene products in human cancer cells has previously been suggested. For example, frizzled-2 (FRZ-2) was originally isolated by Sagara et al., who reported that mRNA from frizzled-2 was not detectable in 15 different normal human adult tissues, with the possible exception of heart tissue, but was found in embryonic tissues, as well as six of eight malignant cell lines (Biochem. Biophys. Res. Comm. 252:117-122 (1998)). However, the ultimate expression of the mRNA and the presence of particular frizzled antigens in cancer cells but not in normal cells has not been described.

There are 18 Wnt and 10 Frizzled genes, all of which are highly homologous in structure, which have been identified thus far from the human genome database (Science, 291:1304-1351 (2001)). However, their high degree of homology and the existence of mRNA encoding these receptors in both normal and tumor tissue would suggest that they would not make a suitable target for immunotherapies.

Despite their homology and widespread existence, it would be expected that there are certain frizzled proteins that are highly specific for tumors. This is because of their involvement in embryogenesis and the hypothesis that many malignant cells may express embryonic patterning receptors. Accordingly, the present invention relates to the design of immunologic compositions and methods that target the portion of the frizzled antigen that is unique to this protein, specific to cancer cells, and also exposed on the cell surface.

### SUMMARY OF THE INVENTION

The present invention relates, inter alia, to a purified antibody for modulating a biological activity of a malignant cell that expresses a frizzled receptor, wherein the antibody specifically binds to at least one epitope in an extracellular domain of the frizzle receptor expressed on the malignant cell. In a preferred embodiment, this extracellular domain comprises the amino terminal peptide fragment of the frizzled receptor. The antibody can further comprises an in-tact antibody or a fragment thereof as described in more detail herein. The purified antibody cal also be capable of sensitizing malignant cells expressing the frizzled receptor to a cytotoxic factor. It is also possible that binding of the antibody to the receptor inhibits binding of the Wnt ligand.

For use in a diagnostic assay, the purified antibody of claim 1 may further comprise a detectable label. In another aspect of the present invention, the antibody may be a human antibody, and may be polyclonal or monoclonal antibody.

In another embodiment, the present invention relates to an isolated nucleic acid, comprising at least one nucleotide fragment encoding an extracellular domain of a frizzled receptor that serves as an epitope for the antibody just described. In instances when it is necessary to enhance the immunogenicity of the frizzled receptor epitope, one can couple the epitope to a known T cell epitope, such as the tetanus toxin. Accordingly, a frizzled receptor epitope conjugate can be prepared comprising at least one epitope in an extracellular domain of the frizzle receptor expressed on a malignant cell and at least one epitope specific to a T cell antigen. It is also possible to enhance the immunogenicity of any given frizzled receptor epitope by preparing a multimer, such as a dimer or trimer, thereof. Such conjugates can be prepared by direct conjugation, or by making use of a linker moiety, such as the GPSL linker.

Another aspect of the present invention relates to a transgenic non-human animal which has been transfected with the nucleic acid encoding the frizzled receptor, or a portion thereof. The present invention also relates to a recombinant vector, comprising at least one nucleic acid encoding the frizzled receptor, or a portion thereof, functionally attached to a promoter region upstream of the nucleic acid. In addition, the present invention relates to a host cell comprising at least one such recombinant vector.

In yet another aspect of the present invention, a pharmaceutical composition is provided which comprises a purified antibody for modulating a biological activity of a malignant cell that expresses a frizzled receptor, wherein the antibody specifically binds to at least one epitope in an extracellular domain of the frizzle receptor expressed on the malignant cell, in a pharmaceutically acceptable carrier.

The present invention also relates to a method for modulating a biological activity of a malignant cell that expresses a frizzled receptor comprising administering a pharmaceutical composition comprising a purified antibody for modulating a biological activity of a malignant cell that expresses a frizzled receptor, wherein the antibody specifically binds to at least one epitope in an extracellular domain of the frizzle receptor expressed on the malignant cell, in a pharmaceutically acceptable carrier.

In a further embodiment, the present invention relates to a pharmaceutical composition useful as a vaccine against malignancy for administration to a patient having a predisposition for the malignancy, wherein the antibody specifically binds to at least one epitope in an extracellular domain of the frizzle receptor expressed on the malignant cell. Such vaccine can be administered using a method of immunizing a subject against a malignancy comprised of malignant cells that express a frizzled receptor, said method comprising the steps of:

a) identifying an antibody for modulating a biological activity of the malignant cell that expresses a frizzled receptor, wherein said antibody specifically binds to at least one epitope in an extracellular domain of the frizzle receptor expressed on the malignant cell; and

b) administering the antibody in a pharmaceutically acceptable carrier in an amount sufficient to inhibit the malignancy.

For use as an immunotherapeutic agent, the present invention relates to a method of treating a subject with a malignancy comprised of malignant cells that express a frizzled receptor, said method comprising the steps of:

a) identifying an antibody for modulating a biological activity of the malignant cell that expresses a frizzled receptor, wherein said antibody specifically binds to at least one epitope in an extracellular domain of the frizzle receptor expressed on the malignant cell; and

b) administering the antibody in a pharmaceutically acceptable carrier in an amount sufficient to modulate a biological activity of the malignant cell.

For use as an immunoassay, the present invention relates to an assay for identifying a frizzled receptor expressed by a malignant cell, wherein said frizzled receptor comprises at least one epitope in an extracellular domain, comprising the steps of:

a) identifying an antibody that specifically binds to the epitope;

b) exposing a sample of cells suspected of expressing the frizzled receptor to the antibody; and

c) determining the extent of binding of the antibody to the cells.

In yet another aspect of the present invention, a screening assay is provided for identification of small molecules that modulate frizzled receptor activity, which comprises:

a) selecting a library of the small molecules comprising a plurality of different chemical structures;

b) contacting the small moleucles with an extracellular domain of a frizzled receptor which is capable of binding to its corresponding Wnt protein; and

c) measuring binding of a ligand to the frizzled receptor in the presence of the small molecule, wherein the ligand is selected from the group consisting of the small molecule, the Wnt protein, and an antibody to the extracellular domain of the frizzled receptor. Such small molecules, which may be nucleic acids, peptides, small organic molecules, or combinations thereof, can function by competing with the Wnt protein for binding to the frizzled receptor, or may mimic the frizzled receptor and bind to the Wnt protein, wherein in the latter instance, the small molecule will prevent binding of both the Wnt protein and an antibody that is specific for the frizzled receptor epitope to which the Wnt protein normally binds from binding thereto. These types of screening methods are well know in the G-protein coupled receptor field, and in particular the field of odorant receptors. See, e.g., U.S. Pat. No. 6,008,000, which discloses assays for screening taste modulating small moleucles that modulate the activity of a G-protein coupled receptor known to be associated with taste.

Other aspects of the present invention are found throughout the specification.

### BRIEF DESCRIPTIONS OF THE DRAWINGS

FIG. 1 depicts a schematic of the developmental signaling pathways.

FIG. 2 depicts the alignment of various deduced amino acid sequences of frizzled receptors derived using the Clustal W program on DeCypher. “CY” refers to the cysteine rich domain. “TM” refers to the transmembrane domain. Accordingly, the regions in-between the CRD and TM domains represent the extracellular regions.

FIG. 3 depicts the sequence alignment of a portion of the first extracellular region of human frizzled receptors.

FIG. 4 depeits the proliferation of SNU1076 cells as described in Example 3

FIGS. 5 and 6 depict the effects of anti-Fz Abs on cancer cell apoptosis as described in Example 4.

FIG. 7 depicts a graphical representation of an olfactory protein, also a G-protein coupled receptor transmembrane protein like the frizzled receptors, showing the amino terminal and three extracellular domain loops, as well as the seven transmembrane domains shown within the cylinders (from PCT WO 92/17585).

FIG. 8 depicts the sequence alignment of the deduced amino acid sequences of human (HFZ) and mouse (MFZ) frizzled receptors 1 to 10, assigned Seq. ID No.s. 44 to 60 in the order shown. Also depicted therein are the amino terminal domains (assigned Seq. ID No.s 61 to 77 in the order shown), the extracellular domain loop 1 (assigned Seq. ID No.s 78 to 94 in the order shown), the extracellular domain loop 2 (assigned Seq. ID No.s 95 to 111 in the order shown), and the extracellular domain loop 3 (assigned Seq. ID No.s 112 to 128 in the order shown.

### DISCLOSURE OF THE INVENTION

The present invention relates to the use of immunologically unique frizzled receptor epitopes as binding targets in the design of compositions and methods that are useful in immunologic based diagnostics and therapeutics of cancers associated with overexpression of frizzled receptors.

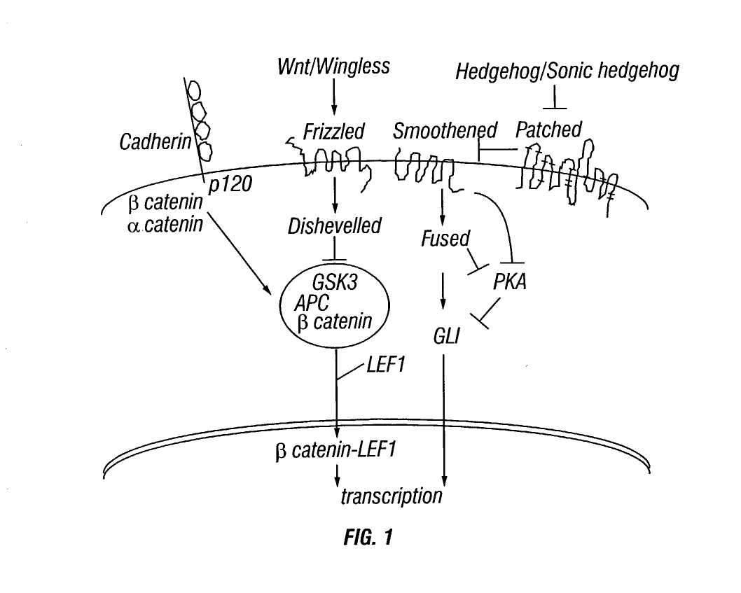

In embryogenesis, body patterning is related to the axial expression of different proteins. The proximal-distal axis is controlled by fibroblast growth factor (FGF), anterior-posterior axis by Sonic hedgehog (SHH), and the dorsal ventral axis by wingless (Wnt). These factors are closely cross-regulated in development. As shown in FIG. 1, the secretion of Wnt is stimulated by SHH signaling and conversely the expression of SHH is supported by the continued presence of Wnt. SHH in turn influences FGF expression. Wnt has been shown to be a ligand for a G-coupled protein receptor in the frizzled (Fz) family of receptors, which mediates a complex signaling cascade. Transcriptional regulation is also mediated by SHH cell surface interaction with its ligand, Patched. Patched tonically inhibits signaling through Smoothened until it binds to SHH. The Wnt/frizzled pathway has been previously implicated in tumorigenesis. Soluble Wnt glycoproteins have been demonstrated to transmit signal by binding to the seven transmembrane domain G-protein coupled-receptor (FIG. 1). Upon Wnt signaling, a cascade is initiated that results in the accumulation of cytoplasmic beta-catenin and its translocation to the nucleus. In the nucleus beta-catenin binds a specific sequence motif at the N terminus of lymphoid-enhancing factor/T cell factor (LEF/TCF) to generate a transcriptionally active complex. Beta-catenin interacts with multiple other proteins such as cadherin which it links to the cytoskeleton. It also associates with the adenomatous polyposis coli (APC) tumor suppressor protein and glycogen synthetase 3 beta (GSK3β). These proteins function to negatively regulate beta catenin by facilitating phosphorylation near the aminoterminus and thus accelerating its proteolytic degradation.

The frizzled receptors are a well-characterized family of transmembrane receptor proteins. To date, there are ten known human frizzled proteins that have been identified from the human genome as follows:

TABLE I
Known Human Frizzled Genes

| Gene | Chromosome | Reference |
|---|---|---|
| FZD1 | 7a21 | Sagara (1988) |
| FZD2 | 17q21.1 | Zhao Z (1995), |
| | | Sagara (1988) |
| FZD3 | 8p21 | Kirikoshi (2000), Sala 2000 |
| FZD4 | 11q14-q21 | Kirikoshi (1999) |
| FZD5 | 2q34 | Wang Y (1996) |
| FZD6 | 8q22.3-q23.1 | Tokuhara (1998) |
| FZD7 | 2q33 | Sagara (1988) |
| FZD8 | 10 | genome |
| FZD9 | 7g11.23 | Wang, YK (1997) |
| FZD10 | 12q24.333 | Koike, et al. (1999) |
| SMOH | 7q31-32 | Stone (1996) |
| | Xie 1998 | |
| FZE3 | This gene could be | Tanaka, et al. (1998) |
| | the same as FZD7 | |

1. Sagara, Biochem. Biophys. Res. Commun. 252(1): 117-122 (1998)
2. Zhao, Genomics 27(2): 370-373 (1995)
3. Kirikoshi, Biochem. Biophys. Res. Commun. 27(1): 8-14 (2000)
4. Sala, Biochem. Biophys. Res. Commun. 273(1): 27-34 (2000)
5. Kirikoshi, Biochem. Biophys. Res. Commun. 264(3): 955-961 (1999)
6. Wang, J. Biol. Chem., 271(8): 4468-4476 (1996)
7. Tokuhara, Biochem. Biophys. Res. Commun. 243(2): 622-627 (1998)
8. Wang, Hum. Mol. Gen. 6(3): 465-472 (1997)
9. Koike, Biochem. Biophys. Res. Commun. 262(1): 39-43
10. Stone, Nature 384(6605): 129-134 (1996)
11. Tanaka, Proc. Nat. Acad. Sci. 95(17): 10164-10169 (1998)

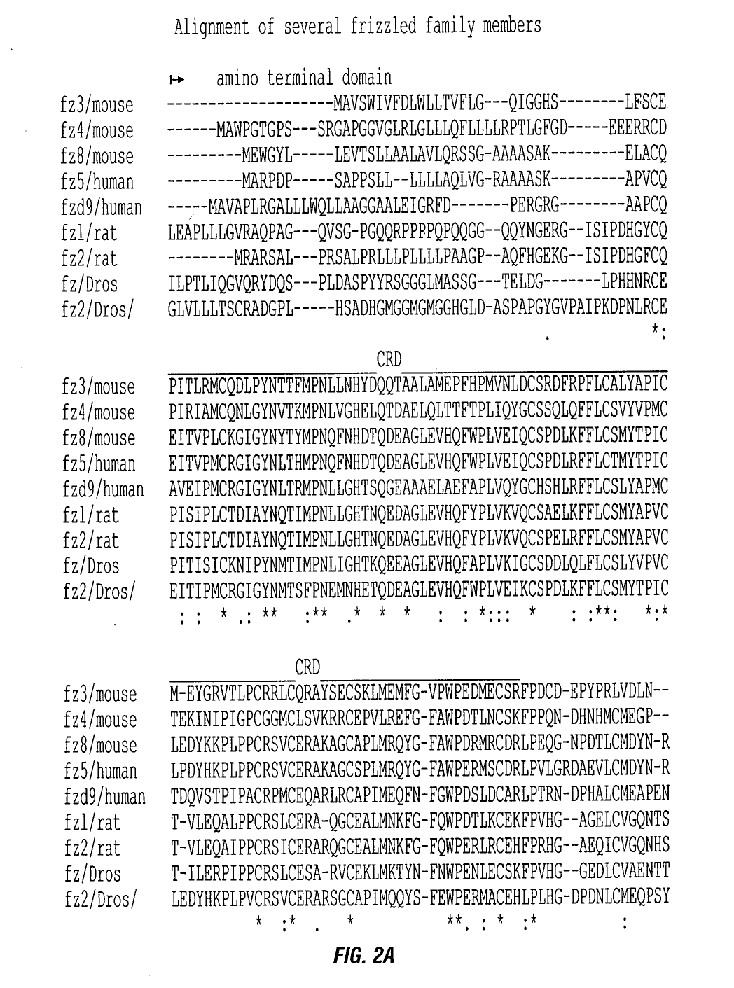

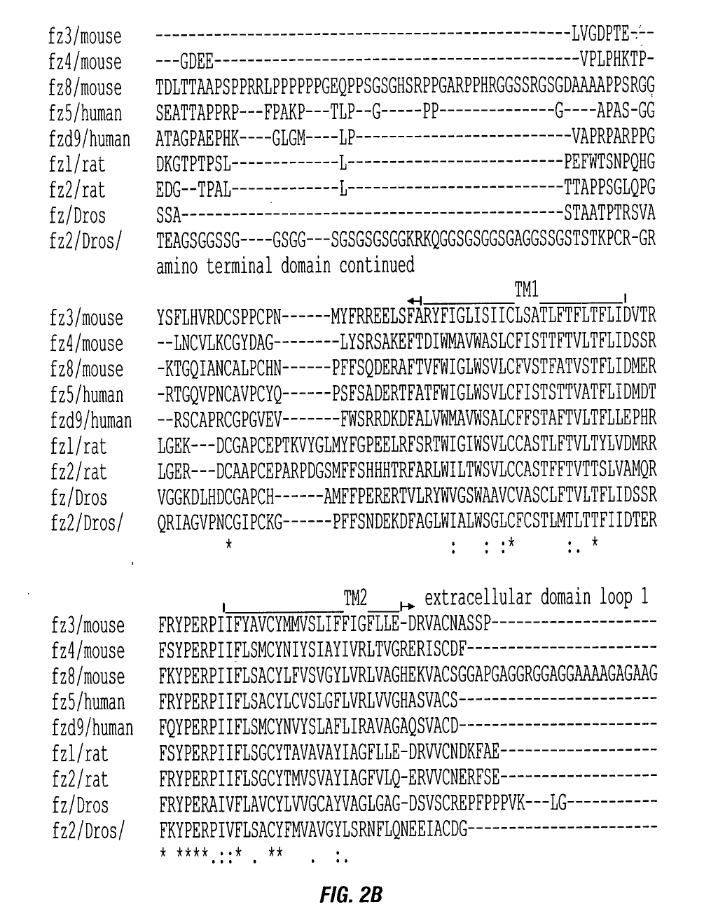

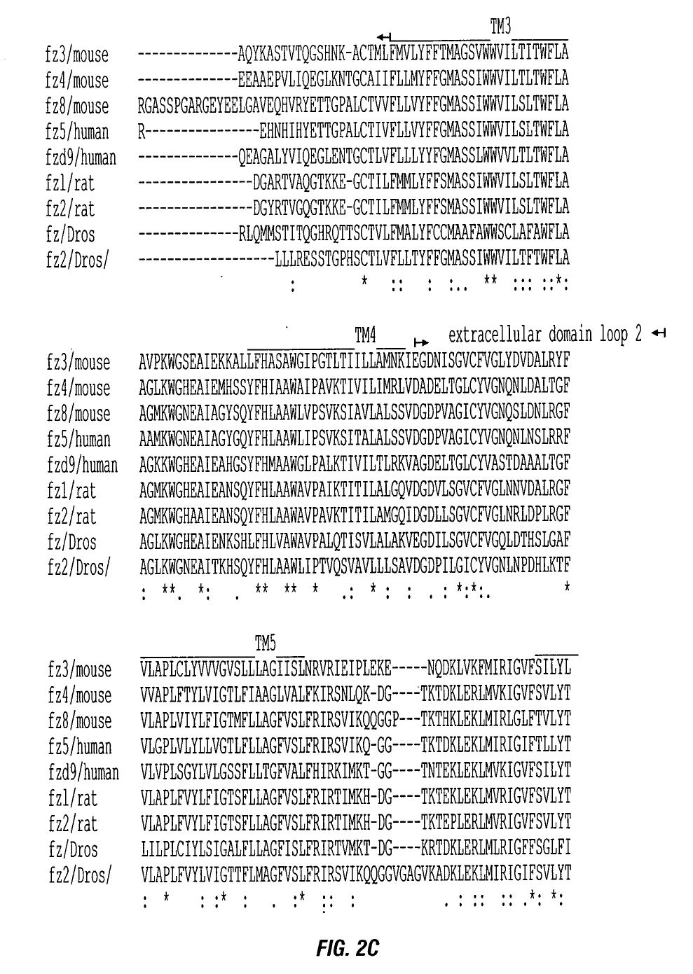

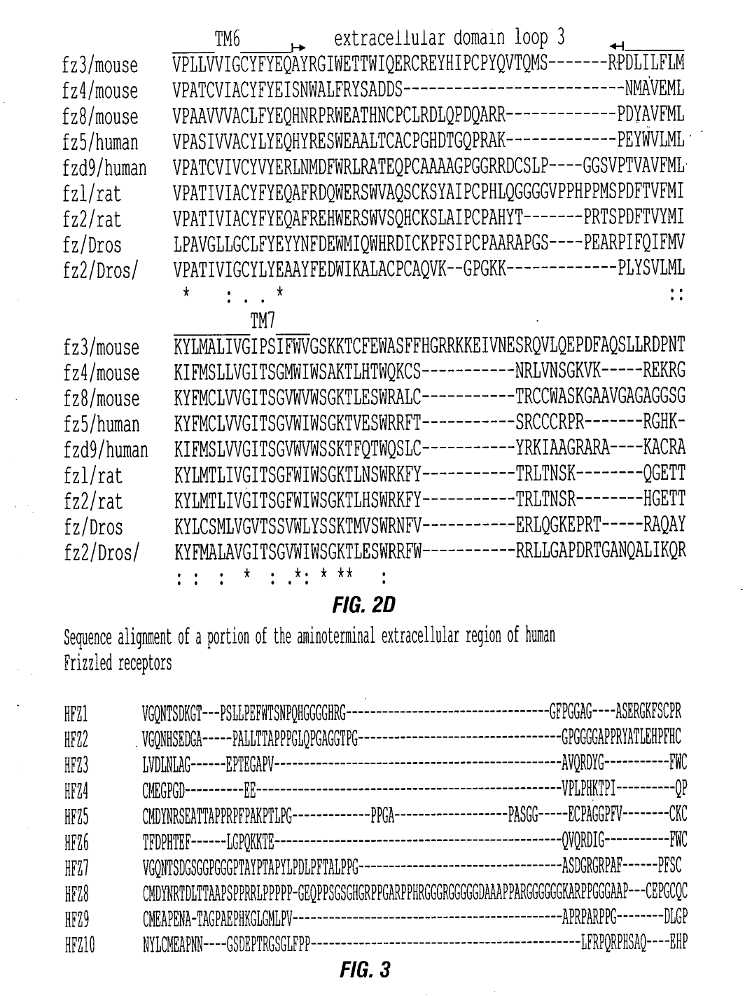

The alignment of several of these frizzled receptors is shown in FIG. 2. As shown therein, Seq. ID No. 35 is assigned to fz3/mouse; Seq. ID NO. 36 is assigned to fz4/mouse; Seq. ID No. 37 is assigned to fz8/mouse; Seq. ID NO. 38 is assigned to fz5/human; Seq. ID No. 39 is assigned to fzd9/human; Seq. ID No. 40 is assigned to fzdl/rat; Seq. ID No. 41 is assigned to fzd2/rat; Seq. ID No. 42 is assigned to fz/Dros; and Seq. ID No. 43 is assigned to fz/Dros.

To evaluate frizzled receptors for their potential as tumor-associated antigens, various hematologic and epithelial tumors are screened by amplifying the mRNA in the tumor cells using a known amplification method, such as reverse-transcription-polymerase chain reaction (RT-PCR) using primers that are specific for known frizzled receptor-associated sequences. From the results of this initial screening, subregions of the nucleic acid sequence are identified that encode the extracellular regions of the frizzled receptor and are further amplified. The sequence alignment of a portion of the first extracellular region is shown in FIG. 3. This extracellular amino terminal domain is generally regarded as antigenic, because of its size and ternary structure.

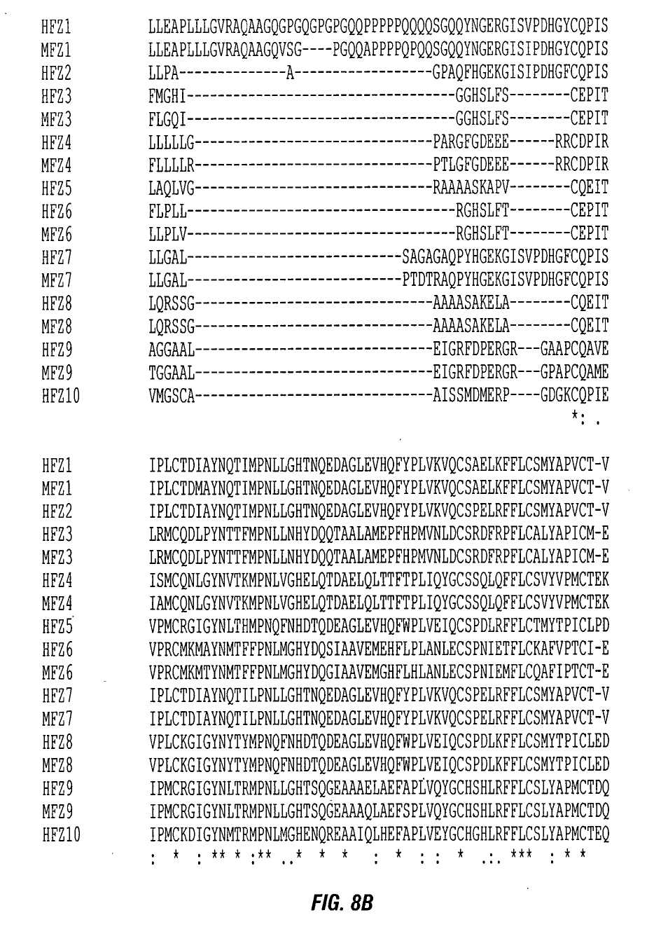

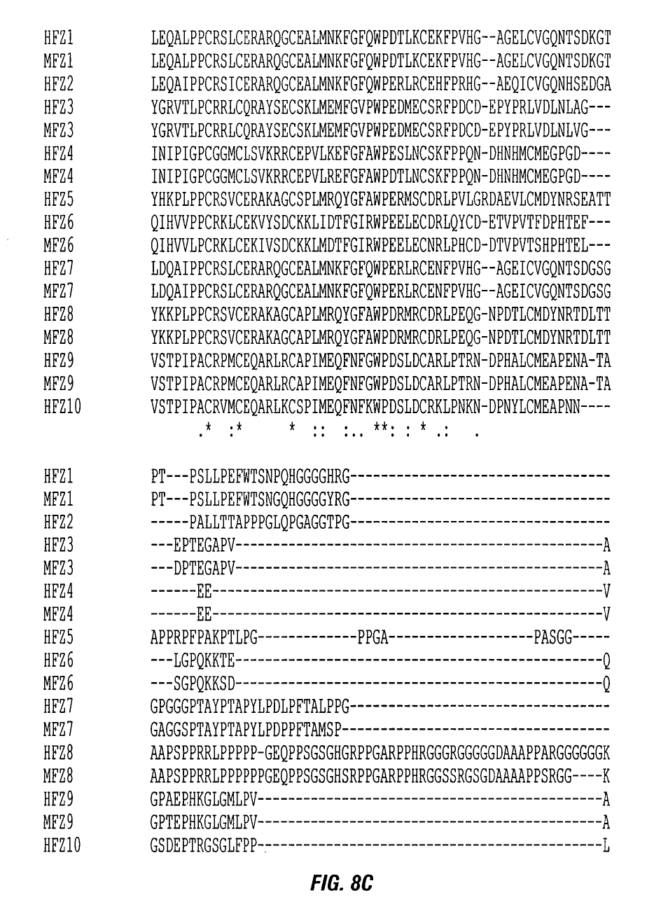

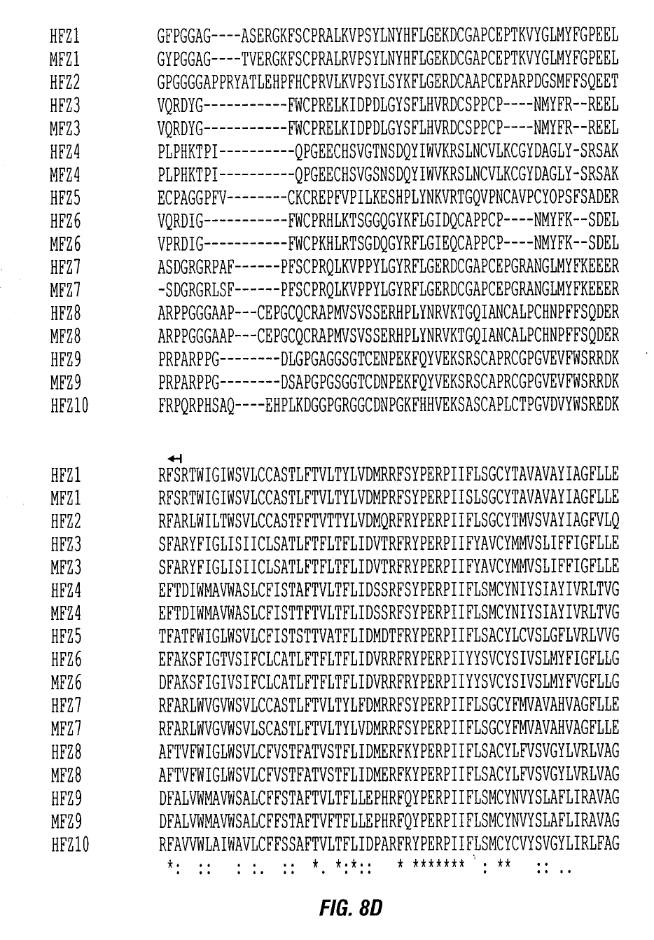

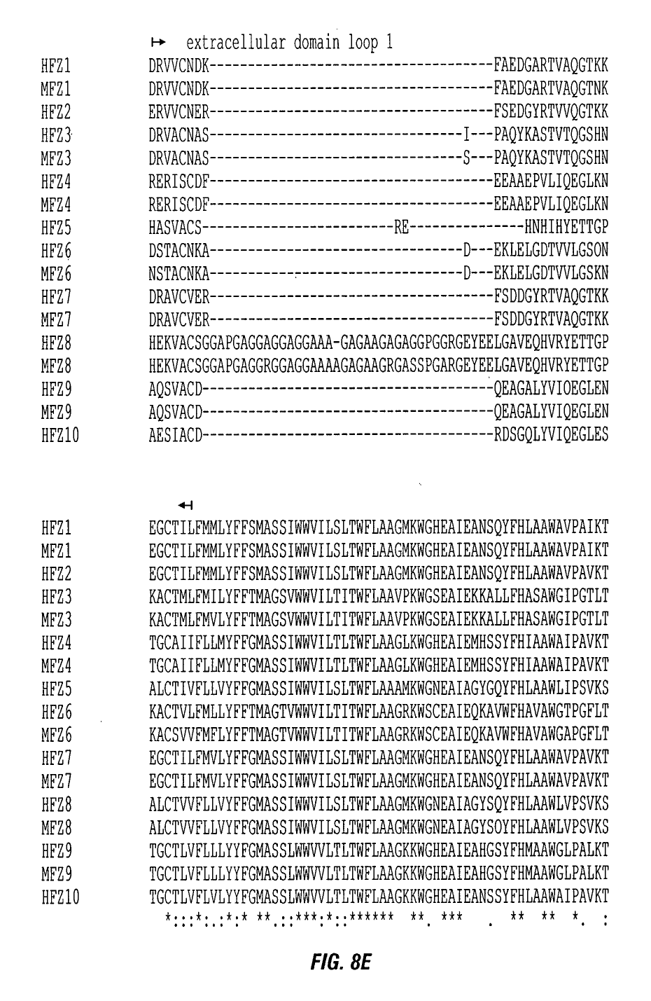

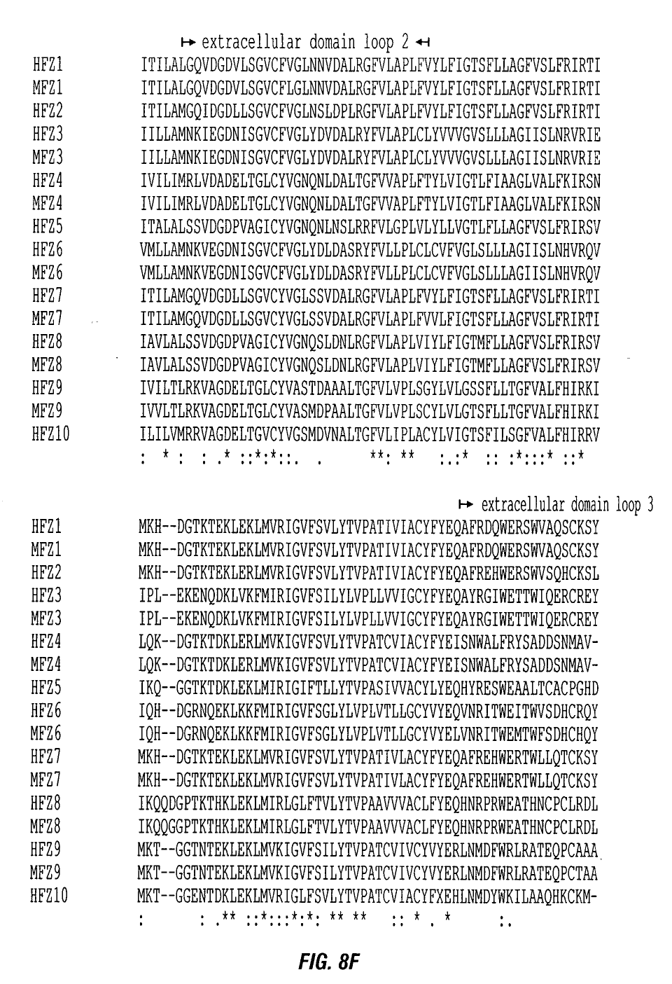

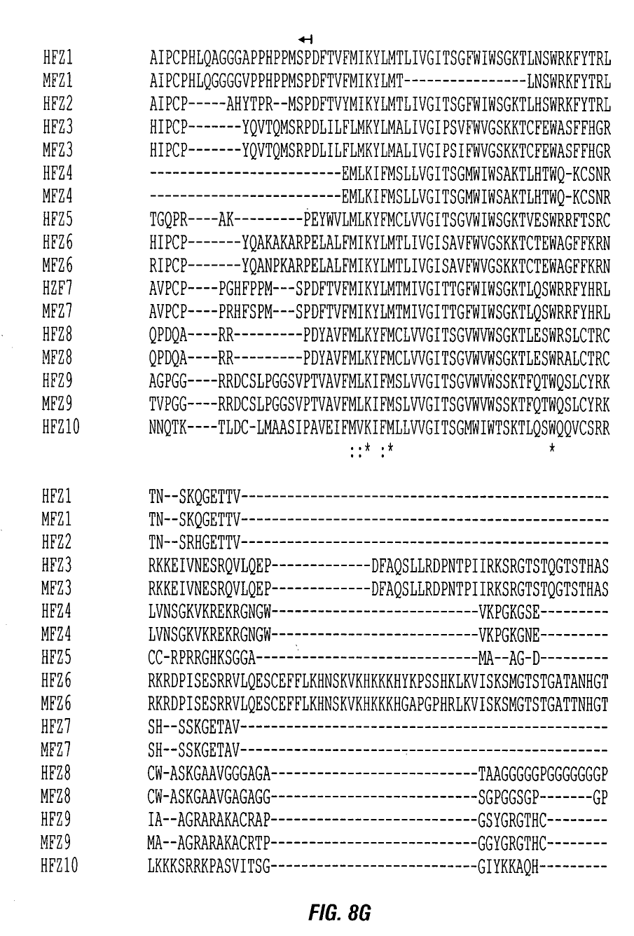

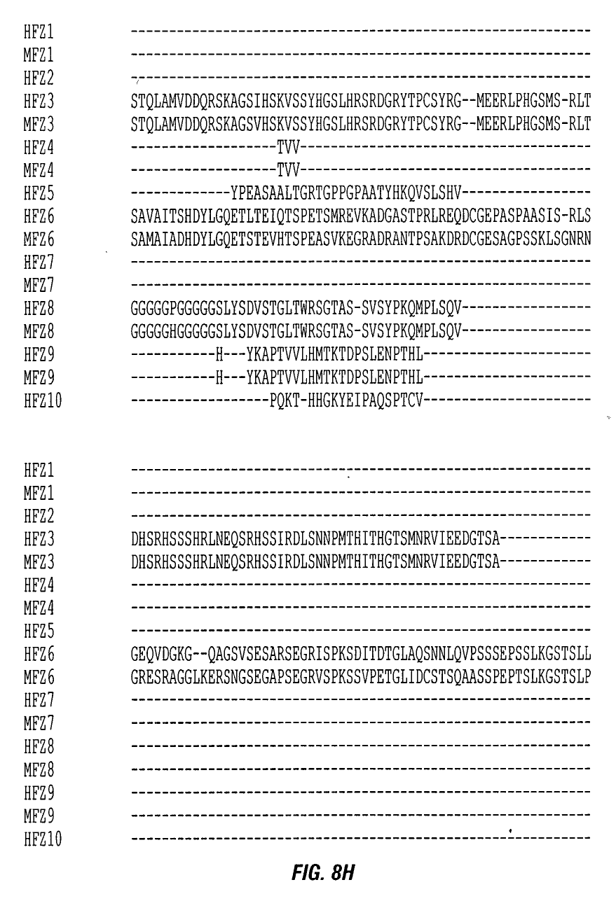

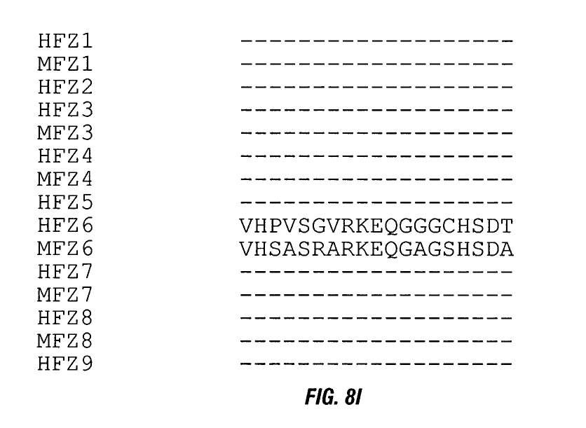

As mentioned elsewhere herein, the gene sequences of frizzled receptors 1 to 10 are known. Also, as shown in FIG. 8, the sequence alignment of the deduced amino acid sequences of human (HFZ) and mouse (MFZ) frizzled receptors 1 to 10 have been determined, and assigned Seq. ID No.s. 44 to 60 in the order shown. Also depicted therein are the amino terminal domains (assigned Seq. ID No.s 61 to 77 in the order shown), the extracellular domain loop 1 (assigned Seq. ID No.s 78 to 94 in the order shown), the extracellular domain loop 2 (assigned Seq. ID No.s 95 to 111 in the order shown), and the extracellular domain loop 3 (assigned Seq. ID No.s 112 to 128 in the order shown.) For example, Seq. ID No. 95 corresponds to the extracellular domain loop 2 for the HFZ1 receptor shown in FIG. 8c, which is:

Seq. ID No. 95: GQVDGDVLSGVCFVGLNNVDALRGF

For convenience, the Seq. ID No. assignments to the human extracellular domains given in FIG. 8 are shown below in Table II:

TABLE II
Sequence ID No.s of Extracellular Domains of
Human Receptors given in FIG. 8

| Frizzled # | Entire Seq. | Amino Terminal | Loop 1 | Loop 2 | Loop 3 |
|---|---|---|---|---|---|
| 1 | 44 | 61 | 78 | 95 | 112 |
| 2 | 46 | 63 | 80 | 97 | 114 |
| 3 | 47 | 64 | 81 | 98 | 115 |
| 4 | 49 | 66 | 83 | 100 | 117 |
| 5 | 51 | 68 | 85 | 102 | 119 |
| 6 | 52 | 69 | 86 | 103 | 120 |
| 7 | 54 | 71 | 88 | 105 | 122 |
| 8 | 56 | 73 | 90 | 107 | 124 |
| 9 | 58 | 75 | 92 | 109 | 126 |
| 10 | 60 | 77 | 94 | 111 | 128 |

1. Primers

A primer is preferably single stranded for maximum efficiency, but may alternatively be in double stranded form. If double stranded, the primer is first treated to separate it from its complementary strand before being used to prepare extension products. Preferably, the primer is a plydeoxyribonucleotide. The primer must be sufficiently long to prime the synthesis of extension products in the presence of the agents for polymerization. The exact lengths of the primers will depend on many factors, including temperature and the source of primer.

Exemplary primer pairs for known human frizzled genes are shown below in Table II.

Table II
PCR Primers for Known Human Frizzled Isoforms

| | | Forward Primers 5′>3′ |
|---|---|---|
| Seq. ID 1 | Frizzled 1 | CCCAGAGCTGCAAGAGCTAC |
| Seq. ID 2 | Frizzled 2 | GCCGTGCCGCTCTATCTGTGAG |
| Seq. ID 3 | Frizzled 3 | ATAGGCCTGATCATCTGAATCTCCTTCA |
| Seq. ID 4 | Frizzled 4 | AACCTCGGCTACAACGTGAGACCAAGAT |
| Seq. ID 5 | Frizzled 5 | ATCGGCTACAACCTGACGCACA |
| Seq. ID 6 | Frizzled 6 | TCTGGAATGTTCACCAAACATTGAAACT |
| Seq. ID 7 | Frizzled 7 | CTCATGAACAAGTTCGGCTTCCAGT |
| Seq. ID 8 | Frizzled 8 | GATGAGGATGAGAGTGAGGTGACATCC |
| Seq. ID 9 | Frizzled 9 | CACGCGCTGTGCATGGAG |
| Seq. ID | Frizzled 10 | CATGGAGGCGCCCAACAAC |
| 10 | | |
| | | Reverse Primers 5′>3′ |
| Seq. ID | Frizzled 1 | CACGATCAGCGTCATAAGGT |
| 11 | | |
| Seq. ID | Frizzled 2 | GTGGCGCGGGAAGTGCTC |
| 12 | | |
| Seq. ID | Frizzled 3 | TCTTGGCACATCCTCAAGGTAATAGGTT |
| 13 | | |
| Seq. ID | Frizzled 4 | GTACTGGATGAGCGGTGTGAAAGTTGT |
| 14 | | |
| Seq. ID | Frizzled 5 | ATGGGCGTGTACATAGTGCATAGGAAG |
| 15 | | |
| Seq. ID | Frizzled 6 | TTTCTCATAAAGTTTACGACAAGGTGGA |
| 16 | | |
| Seq. ID | Frizzled 7 | CGCGGTAGGGTAGGCAGTGG |
| 17 | | |
| Seq. ID | Frizzled 8 | ACTCAGACTTCCTGGCTCTCAGGTG |
| 18 | | |
| Seq. ID | Frizzled 9 | GGCTCTTCTCCACGTACTGGAACTTCT |
| 19 | | |
| Seq. ID | Frizzled 10 | GTCCTTCAGCGGGTGCTCCT |
| 20 | | |

The primers described herein are selected to be “substantially” complementary to the different strands of each specific sequence to be synthesized or amplified. This means that the primer must be sufficiently complementary to hybridize relatively specifically with its intended primer site in the target template strand. Therefore, the primer sequence may or may not reflect the exact sequence of the template. For example, a non-complementary nucleotide fragment can be attached to the 5′ end of the primer, with the remainder of the primer sequence being substantially complementary to the strand. Such non-complementary fragments typically contain an endonuclease restriction site. Alternatively, non-complementary bases or longer sequences can be interspersed into the primer, provided the primer sequence has sufficient complementarity overall with the sequence of the strand to be synthesized or amplified to non-randomly hybridize therewith and thereby form an extension product under polynucleotide synthesizing conditions.

A frizzled gene-specific primer preferably includes at least about 15 nucleotides, more preferably at least about 20 nucleotides. The primer preferably does not exceed about 30 nucleotides, more preferably about 25 nucleotides, although it can contain fewer nucleotides. Short primer molecules generally require lower temperatures to form sufficiently stable hybrid complexes with the template. Most preferably, the primer includes between about 20 to about 25 nucleotides. The length of the primer will vary inversely with the extent of conservation of the complementary exon sequence. The GC content of the primers should be about 50%.

Primers can be prepared using a number of methods, including phosphotriester and phosphodiester methods or automated embodiments thereof. The phosphodiester and phosphotriester methods are described in Cruthers, Science, 230:281-285 (1985); Brown et al., Meth. Enzymol., 68:109 (1979); and Nrang et al., Meth. Enzymol., 68:90 (1979). In one automated method, diethylphosphoramidites which can be synthesized as described by Beaucage et al., Tetrahedron letters, 22:1859-1962 (1981) are used as starting materials. A method for synthesizing primer oligonucleotide sequences on a modified solid support is described in U.S. Pat. No. 4,458,066.

Primer extension reactions are preferably performed using purified DNA from the target organism. Isolation of DNA from cells is routine in the art and there are numerous sources of nucleic acid isolation protocols suited for microorganisms such as bacteria and fungi including mammalian cells (e.g., Sambrook et al., supra, (1989)). Primer extension reactions also can be performed using DNA that has not been purified but is accessible to the primer. The DNA can be accessible naturally in the sample or can be made accessible following one or more processing steps.

2. Amplification

The frizzled gene amplifying primers are used to amplify products from tumor cells in a primer extension reaction. A variety of primer extension reactions can be used with the present methods. Non PCR amplification methods include ligase chain reaction (LCR: Barany et al., PCR Meth. Applic., 1:15-16 (1991)), self-sustained sequence replication (SSR: Muller et al., Histochem. Cell Biol., 108:431-437 (1997)), also known as nucleic acid sequence-based amplification: NASBA) and its new derivative, cooperative amplification of templates by cross-hybridization (CATCH: Ehricht et al., Eur. J Biochem., 243:358-364 (1997)), transcript-based amplification system (AMPLISCRIPT®, Kaylx Biosciences, Nepean, Ontario Canada), replicatable RNA reporter systems based on the Q beta replicase, hybridization-based formats such as strand-displacement amplification (SDA: Becton-Dickinson, Franklin Lakes, N.J.; Walker et al. Nucleic Acids Res., 20:1691-1696 (1992)), and chip-based microarrays such as Affymetrix GeneChip (Fodor et al., Nature, (Lond) 364:555-556 (1993)).

Signal amplification methods also can be used to enhance detectability such as with the use of compound probes (Fahrlander et al., Bio/Technology, 6:1165-1168 (1988)) or branched probes (Chiron Corp., Emeryville, Calif.; Urdea et al., Nucleic Acids Symp. Ser., 24:197-200 (1991)) as is well known in the art.

Primer extension by PCR is performed by combining one or more primers with the target nucleic acid and a PCR buffer containing a suitable nucleic acid polymerase. The mixture is thermocycled for a number of cycles, which is typically predetermined, sufficient for the formation of a PCR reaction product, thereby enriching the sample to be assayed for the sequence of interest. Protocols for PCR are well known in the art (e.g., U.S. Pat. Nos. 4,683,192, 4,683,202, 4,800,159, and 4,965,188) and are available from a variety of sources (e.g., PCR Technology: Principles and Applications for DNA Amplification, H. Erlich, ed., Stockton Press, New York (1989); and PCR Protocols: A Guide to Methods and Applications, Innis et al., eds., Academic Press, San Diego, Calif. (1990)).

PCR is typically carried out by thermocycling, i.e., repeatedly increasing and decreasing the temperature of a PCR reaction admixture within a temperature range whose lower limit is about 30 degrees Celsius (30° C.) to about 55° C., and whose upper limit is about 90° C. to about 100° C. Increasing and decreasing the temperature can be continuous, but is preferably phasic with time periods of relative temperature stability at each of the temperatures favoring polynucleotide synthesis, denaturation and hybridization. Thus, the PCR mixture is heated to about 90-100° C. for about 1 to 10 minutes, preferably from 1 to 4 minutes. After this heating period, the solution is allowed to cool to about 54° C., which is preferable for primer hybridization. The synthesis reaction may occur at room temperature up to a temperature above which the polymerase (inducing agent) no longer functions efficiently. Thus, for example, if Taq DNA polymerase is used as inducing agent, the temperature is generally about 70° C. The thermocycling is repeated until the desired amount of amplified product is produced.

A single frizzled gene-specific primer pair can be used in each amplification reaction. Alternatively, additional primers from other primers pairs can be included in the reaction. The primers are generally added in molar excess over template DNA. The conditions of the PCR are adjusted depending on a number of factors, including the degree of mismatch, the GC content of the primer, the length of the primer factors affecting PCR conditions, melting temperature of the primer, and product length and placement within the target sequence. Adjustments in the concentrations of the reaction components, especially magnesium concentration, can be used to enhance the conditions for PCR.

The PCR buffer contains the deoxyribonucleoside triphosphates (i.e., polynucleotide synthesis substrates) dATP, dCTP, dGTP, and dTTP and a polymerase, typically thermostable, all in amounts sufficient for the primer extension (i.e., polynucleotide synthesis) reaction. An exemplary PCR buffer comprises the following: 50 mM KCl; 10 mM Tris-HCl at pH 8.3; 1.5 mM MgCl₂; 0.001% (wt/vol) gelatin, 200 microMolar (μM) dATP, 200 μM dTTP, 200 μM dCTP, 200 μM dGTP, and 2.5 units Thermus aquaticus (Taq) DNA polymerase I (U.S. Pat. No. 4,889,818) per 100 microliters (μL) of buffer.

The inducing agent may be any compound or system which will function to accomplish the synthesis of primer extension products, including enzymes. Suitable enzymes for this purpose include, for example, E. coli DNA polymerase I, Klenow fragment of E. coli DNA polymerase I, T4 DNA polymerase, other available DNA polymerases, reverse transcriptase, and other enzymes, such as heat-stable enzymes that facilitate combination of the nucleotides in the proper manner to form the primer extension products complementary to each nucleic acid strand. Generally, the synthesis will be initiated at the 3′ end of each primer and proceed in the 5′ direction along the template strand, until synthesis terminates, producing molecules of different lengths. There may be inducing agents, however, which initiate synthesis at the 5′ end and proceed in the above direction, using the same process as described above. Frizzled gene-specific primers suitable for such inducing agents can be designed using the principles elaborated above for inducing agents that extend from the 3′ end.

The PCR reaction can advantageously be used to incorporate into the product a preselected restriction site useful in later cloning and sequencing of the amplified product. This can be accomplished by synthesizing the primer with the restriction site in the 5′ end of the primer.

3. Arrays

In cases where hybridization assays of multiple tumor cell genomes are desired to be performed simultaneously using the same intronic region-specific probes, it would be convenient to perform such hybridizations in an array format. Such assay formats and minaturizations thereof, i.e. microchip assays, are well known in the literature and could easily be adapted for the assays described herein. For example, see PCT WO 00/03037, which describes screening arrays of nucleotides using specific probes. After compilation of the sequences from a variety of tumor cells, these sequences can be used in a microarray format on a microchip to perform simultaneous hybridization studies with various probes or sequences from other tumor cells.

Alternatively, such assay formats can be designed for use to study hybridization of an array of frizzled gene-specific sequences with a single tumor cell genome, or an array of the protein products derived from the translation of the frizzled gene sequences of a population of cells, or an array of antibodies to such protein products, or combinations thereof in two-dimensional arrays. Such microarray hybridization assays can easily be performed using a variety of known microchip assay formats and techniques.

In addition to such arrays, the methods of the present invention can be adapted to an array format to screen small molecule libraries for their ability to modulate the biological activities of metastatic cells. For example, small molecule libraries can be screed as potential ligands for frizzled receptors in an array using the antibodies described herein that bind to the extracellular domains in a competitive (or other) assay format. Small molecules which compete with the antibodies for binding to the frizzled receptor would be candidates for further screening as therapeutic agents, and may include small peptide fragments, nucleic acids or organic compound, or combinations thereof.

Analysis of nucleic acid from known tumor cells or products produced therefrom by primer extension as described herein also can include analysis of the sequence of the amplified frizzled gene of the tumor cell DNA. For example, amplified products such as from a PCR can be directly cloned by a variety of methods well known in the art (e.g., Ausubel et al., Molecular cloning of PCR products, in: Short Protocols in Molecular Biology, 3rd Ed. John Wiley & Sons, Inc., New York, pp. 15-32 (1997)). Cloning of amplified products can be accomplished using “sticky ends” such as the TA cloning method or by “blunt end” cloning approaches. Alternatively, frizzled gene-specific primers can be designed with endonuclease restriction sites at the 5′ end of the primer which are designed for cutting and insertion into a specified cloning vector. Kits are commercially available for cloning amplified products such as produced in a PCR (e.g., Invitrogen, Inc., San Diego, Calif.).

Methods for sequencing genes are well known, including the Sanger dideoxy mediated chain-termination approach and the Maxam-Gilbert chemical degradation approach. These and other nucleic acid sequencing methods are described, for example, in Sambrook et al., supra, (1989) (chapter 13). Nucleic acid sequencing can be automated using a number of commercially available instruments.

Amplified products also can be directly sequenced without cloning the product (e.g., Sambrook et al., supra, (1989) (14.22-14.29)). Amplified products that have been purified, for example, by gel electrophoresis, are suitable for direct sequencing (id.).

4. Antibodies

The present invention relies on the ability to design antigen-antibody binding pairs using the extracellular domains of the frizzled receptor as the antigenic epitope. Such antibodies are useful for detecting tumor-specific frizzled receptor epitopes, as well as for imnimunotherapy of cancers. Although many regions of the extracellular domain may have sufficient size and tertiary structure to be independently antigenic, others may require coupling to T helper epitopes. This can be achieved using techniques that are well known in the art (e.g., Harlow and Lane, “Antibodies: A laboratory Manual,” Cold Spring Harber Laberatory Press (1988)). Although there are numerous T helper epitopes known in the art, tetanus toxin and measles virus fusion (MVF) protein T helper epitopes are exemplary. As used herein, the term “frizzled epitope” refers both to an independently antigenic extracellular domain of a frizzled receptor, as well as one which is coupled to a T helper epitope to enhance immunogenicity.

An anti-frizzled epitope antibody (“anti-Fz Ab”) is used in its broadest sense to include polyclonal and monoclonal antibodies, as well as polypeptide fragments of antibodies that retain a specific binding affinity for its target antigen of, e.g., at least about 1×10⁵M⁻¹. One skilled in the art would know that antibody fragments such as Fab, F(ab′)₂ and Fv fragments can retain specific binding activity for their target antigen and, thus, are included within the definition of an antibody herein. In addition, the term “antibody” as used herein includes naturally occurring antibodies as well as non-naturally occurring antibodies such as domain-deleted antibodies (Morrison et al., WO 89/07142 ) or single chain Fv (Ladner et al., U.S. Pat. No. 5,250,203). Such non-naturally occurring antibodies can be constructed using solid phase peptide synthesis, can be produced recombinantly or can be obtained, for example, by screening combinatorial libraries consisting of variable heavy chains and variable light chains using known methods, such as those described by Huse et al., Science, 246:1275-1281 (1989).

Antibodies to frizzled epitopes can be prepared using a substantially purified extracellular region of a frizzled receptor, or a fragment thereof, which can be obtained from natural sources or produced by recombinant DNA methods or chemical synthesis. For example, recombinant DNA methods can be used to express the frizzled gene sequence alone or as a fusion protein, the latter facilitating purification of the antigen and enhancing its immunogenicity.

Antisera containing polyclonal antibodies reactive with antigenic epitopes of the frizzled receptor can be raised in rabbits, goats or other animals. The resulting antiserum can be processed by purification of an IgG antibody fraction using protein A-Sepharose chromatography and, if desired, can be further purified by affinity chromatography using, for example, Sepharose conjugated with a peptide antigen. The ability of polyclonal antibodies to specifically bind to a given molecule can be manipulated, for example, by dilution or by adsorption to remove crossreacting antibodies to a non-target molecule. Methods to manipulate the specificity of polyclonal antibodies are well known to those in the art (e.g., Harlow and Lane, supra, (1988)).

A monoclonal antibody specific for the frizzled eptope can be produced using known methods (Harlow and Lane, supra, (1988)). Essentially, spleen cells from a mouse or rat immunized as discussed above are fused to an appropriate myeloma cell line such as SP2/0 myeloma cells to produce hybridoma cells. Cloned hybridoma cell lines can be screened using a labeled frizzled epitope to identify clones that secrete an appropriate monoclonal antibody. A hybridoma that expresses an antibody having a desirable specificity and affinity can be isolated and utilized as a continuous source of monoclonal antibodies. Methods for identifying an anti-Fz Ab having an appropriate specificity and affinity and, therefore, useful in the invention are known in the art and include, for example, enzyme-linked immunoadsorbence assays, radioimmunoassays, precipitin assays and immunohistochemical analyses (e.g., Harlow and Lane, supra, (1988) (chapter 14)).

An anti-Fz Ab can be characterized by its ability to bind specifically to the cells that express the particular frizzled receptor. In addition, an anti-Fz Ab of the invention can be used to purify frizzled receptors from a biological or experimentally prepared sample. For example, such antibodies can be attached to a solid substrate such as a resin and can be used to affinity purify the frizzled receptor. In addition, the anti-Fz Ab can be used to identify the presence of the frizzled receptor in a sample. In this case, the antibody can be labeled with a detectable label such as a radioisotope, an enzyme, a fluorochrome or biotin. An anti-Fz Ab can be detectably labeled using methods well known in the art (e.g., Harlow and Lane, supra, (1988) (chapter 9)). Following contact of a labeled anti-Fz Ab with a sample, specifically bound labeled antibody can be identified by detecting the label.

5. Immunoassays

The binding of an anti-Fz Ab to the frizzled receptor also can be determined using immunological binding reagents. As used herein, an immunological binding reagent includes any type of biomolecule that is useful to detect an antibody molecule. An immunological binding reagent can include a labeled second antibody. A second antibody generally will be specific for the particular class of the first antibody. For example, if an anti-frizzled epitope antibody (i.e., a first antibody) is of the IgG class, a second antibody will be an anti-IgG antibody. Such second antibodies are readily available from commercial sources. The second antibody can be labeled using a detectable moiety as described above. When a sample is labeled using a second antibody, the sample is first contacted with a first antibody (i.e., anti-Fz Ab), then the sample is contacted with the labeled second antibody, which specifically binds to the first antibody and results in a labeled sample. Alternatively, a labeled second antibody can be one that reacts with a chemical moiety, for example biotin or a hapten that has been conjugated to the first antibody (e.g., Harlow and Lane, supra, (1988) (chapter 9)). Immunological binding agents also can include avidin or streptavidin when the anti-frizzled epitope antibody is labeled with biotin.

Principally, all conventional immunoassays are suitable for the detection of frizzled receptors. Direct binding as discussed above or competitive tests can be used. In a competitive test, the anti-Fz Ab can be incubated with a sample and with the frizzled receptors or a fragment thereof (produced as described herein) both simultaneously or sequentially. The frizzled receptors from the sample preferably competes with the added frizzled epitope (hapten) of the invention for the binding to the antibody, so that the binding of the antibody to the hapten in accordance with the invention is a measure for the quantity of antigen contained in the sample. In a heterogeneous competitive immunoassay where the liquid phase is separated from the solid phase, both the antibody or the peptide can be labeled or bound to a solid phase. The exact amount of antigen contained in the sample can then be determined in a conventional manner by comparison with a standard treated in the same manner.

All competitive test formats that are known to the expert can be used for the detection. The detection can be carried out, for example, using the turbidimetric inhibition immunoassay (TINIA) or a latex particle imnmunoassay (LPIA). When a TINIA is used, the peptide or peptide derivative of the invention is bound to a carrier such as dextran (EP-A-0 545 350). This polyhapten competes with the analyte contained in the sample for the binding to the antibody. The formed complex can be determined either turbidimetrically or nephelometrically. When an LPIA is employed, particles, preferably latex particles, are coated with the peptides of the invention and mixed with the antibody of the invention and the sample. When an analyte is present in the sample, agglutination is reduced.

Enzyme immunoassays (Wisdom, Clin. Chem., 22(8):1243-1255 (1976), and Oellerich, J. Clin. Chem. Clin. Biochem., 18:197-208 (1980)), fluorescence polarization immunoassays (FPIA) (Dandliker et al., J Exp. Med., 122:1029 (1965)), enzyme-multiplied immunoassay technology (EMIT) (Rubenstein, Biochem. Biophys. Res. Comm., 47:846-851 (1972)) or the CEDIA technology (Henderson et al., Clin. Chem., 32:1637-41 (1986)) also are suitable immunological based assays for detection of frizzled receptors.

6. Immunotherapeutics

One aspect of the present invention is the design of immunotherapies for cancer. Wnt signaling through frizzled receptors has been described to inhibit apoptosis. Also, some of the genes that are regulated by TCF/beta-catenin are known to be associated with the cell cycle and cell proliferation. By blocking the binding of Wnt proteins to their receptors via antibodies directed to the extracellular portion of frizzled receptors, this pathway can be interrupted. Thus, it is believed that disruption of the downstream translocation of beta-catenin to the nucleus results in slower tumor growth or death of the cell.

As used herein, the term “modulating a biological activity of a malignant cell” refers to the ability of the antibody to effect cellular function. These effects may manifest themselves as cell growth inhibition, the ability to elicit a cytotoxic response to the malignant cell, or other such negative effects on the malignancy. Although not wishing to be bound to any particular theory, it is believed that this effect is caused by the antibody binding to the extracellular domain of the frizzled receptor in a way that interferes with the Wnt/frizzled signalling pathway.

The pharmaceutical compositions of the present invention include therapeutically effective amount of the appropriate anti-Fz Ab in a pharmaceutically acceptable carrier. Such carriers are well known in the art. Examples of appropriate carriers are those that are known for delivery of interferons, such as normal saline, dextrose, etc. The mode of administration of the pharmaceutical composition necessarity depends on the type and location of the target tumor cells. Accordingly, the compositions can be delivered, e.g., parenterally, or typically intraveneously in a solution, suspension or emulsion.

Pharmaceutical compositions and rouths of administration of aqueous compositions comprise an effective amount of the antibody in the pharmaceutically acceptable carrier. By “pharmaceutically acceptable” it is intended that the compositions do not produce adverse, allergic reactions when administered to the animal or human subject, and such carriers include solvents, dispersion media, coatings and the like. Excipients may also be added, which include, inter alia, antimicrobial agents, isotonicity enhancers, absorption delaying agents, surfactants, dispersants, preservatives, and the like.

For administration to an animal or human subject, the solutions are necessarily prepared to meet all FDA Office of Biologics standards. As such, they are normally dialozed to remove undesired small molecular weight molecules or lyophilized with other active and excipient ingredients for reconstitution prior to administration. As would be appreciated by one of skill in the art, the administration parameters, such as dosage and timing, will necessarily depend on the type and location of the metastes to be treated and would easily be determined using routine optimization principles based on other like immunotherapeutics. Routes of suitable administration may include injection, intraveneous, intramuscular, subcutaneous, intralesional, and the like. Alternatively, the immunotherapeutics of the present invention can be formulated for other local routes of administration as topicals, inhalants, orthotopic, ophthalmic, and the like.

An “effective” amount of the immunotherapeutics of the present invention is, of course, determined based on the intended therapeutic goal. As such, a “dosage” of the therapeutic refers to the unit amount of the therapeutic expected to achieve the desired goal, each unit containing a predetermined quantity of the therapeutic to be administered by the appropriate route of administration. Administration may also be spaced out over time to maximize the therapeutic effect, such as two to six administrations spaced out in intervals of several hours to several weeks.

The course of treatment may be monitoried using appropriate immunoassays. For example, the level of circulating anti-Fz Abs following administration can easily be monitored using labeled anti-immunoglobulin antibodies in any of a number of commercially available assay formats.

### EXAMPLES

The following examples are included for illustrative purposes only and are not intended to limit the scope of the invention.

### Example 1

### Expression of Frizzled Gene mRNA From Normal and Cancer Cells

To evaluate frizzled receptors for their potential as tumor associated antigens, the mRNA from various hematologic and epithelial tumors were screened, as well as the MRNA from normal cell lines. In this example total RNA was extracted from HNSCC lines (PCI13, Detroit 562, RPMI 2650, SNU1076, KB, AMC4), a CLL line (Lesch), a Burkitt lymphoma line (Ramos), glioma lines (U87MG, and U373MG), normal human bronchial epithelial cell lines (Clonetics, San Diego, Calif.) and normal oral squamous epithelial (OSE) cells using Trizol® (Gibco, BRL, Grand Island, N.Y.). Reverse transcription was performed using 1□ g of RNA from each sample and the Superscript™ Preamplification kit (Gibco BRL). Different pairs of gene-specific primers based on sequences of cloned human isoforms of the frizzled genes were used for reverse transcriptase-PCR (RT-PCR) analysis.

The following list summarizes the primer pairs used:

| | | | |
|---|---|---|---|
| FZD2 | (Seq. ID 21): | 5′-cagcgtcttgcccgaccagatcca-3′ | (reverse); |
| | (Seq. ID 22) | 5′-ctagcgccgctcttcgtgtacctg-3′ | (forward). |
| FZD5: | (Seq. ID 23) | 5′-ttcatgtgcctggtggtgggc-3′ | (forward); |
| | (Seq. ID 24) | 5′-tacacgtgcgacagggacacc-3′ | (reverse) |
| G3PDH: | (Seq. ID 25) | 5′-accacagtccatgccatcac-3′ | (forward); |
| | (Seq. ID 26) | 5′-tacagcaacagggtggtgga-3′ | (reverse). |

Frizzled 2 was amplified with 25 cycles of PCR. Frizzled 5 and G3PDH were amplified with 30 cycles of PCR. The amplification products for frizzled 2 and G3PDH are shown. The expression of the frizzled isoforms in cancer cells was confirmed by sequencing.

In an expanded cell set total RNA was extracted from 14 tumor cell lines, two normal human bronchial epithelial cell lines and 10 normal oral mucosal epithelial cells by using Trizol™. Cancer cell lines consisted of 10 head and neck squamous cell cancers (HNSCC), 2 B-cell tumor cell lines, and 2 glioma cell lines. Two normal human bronchial epithelial cell samples were purchased from Clonetics (San Diego, Calif.). Ten normal oral mucosal cell samples (Oral SC) were obtained from scraping the oral mucosa from 10 volunteers. RT-PCR analysis was performed as described above. These results are shown in Table III below.

TABLE III
:Summary of frizzled genes detected by RT-PCR
in normal and cancer cells

| | Normal (11) | | Cancer (14) | | |
|---|---|---|---|---|---|
| mRNA | Oral SC | NHBE | Glioma | HNSCC | B cell tumor |
| amlified | (10) | (2) | (2) | (10) | (2) |
| Frizzled 2 | 0 | 1 | 2 | 10 | 2 |
| Frizzled 5 | 4 | 1 | 1 | 9 | 1 |

As shown, in some instances, the frizzled gene associated mRNA is expressed in overabundance in cancer cells when compared to normal cells.

### Example 2

### Analysis of Frizzled 2 Protein Expression in Cancer vs. Normal Cells

To determine the amount of protein expressed in the cells studied in Example 1, adherent cells in culture were harvested and lysed with a solution containing 25 mM Tris HCl, 150 mM KCl, 5 mM EDTA, 1% NP-40, 0.5% sodium deoxycholic acid, 0.1% sodium dodecyl sulfate,1 mM NaVO₃, 1 mM NaF, 20 mM □-glycerophosphate and protease inhibitors. Twenty μg of protein from each cell line was separated by SDS-PAGE and transferred to a PVDF membrane. The membrane was immersed in 2% I-block, 0.05% Tween X in PBS and then incubated with a 1:500 dilution of polyclonal goat anti-human frizzled 2 IgG (Santa Cruz Biotechnology, Santa Cruz, Calif.). These primary antibodies were then detected by horseradish peroxidase-conjugated donkey anti-goat IgG (Santa Cruz) and chemiluminiscence (ECL detection reagents, Amersham Life Science, Aylesbury, UK). To verify relative amount of protein transferred in each lane, the presence of actin was measured with an actin monoclonal antibody (Chemicon International Inc, Temecula, Calif.).

The result of this experiment (not shown) revealed that, although frizzled 2 associated mRNA was detected by RT-PCR as shown in Table III, no detectable amount of protein was detectable immunologically. These contrasting results indicate that tumor specificity of the frizzled receptors at the protein level cannot accurately be predicted by looking at tumor specificity at the mRNA level.

### Example 3

### The Effects of Anti-Fz Abs on Cancer Cell Growth

The ability to block the Wnt-frizzled signaling pathway can provide an effective way of limiting growth of tumor cells. In order to determine the efficacy of using such anti-Fz Abs as an adjunctive passive immunotherapy, such as that observed using humanized anti-HER2 antibodies (Herceptin, Genentech, inc., South San Francisco, Calif.), the effects of anti-frizzled 2 antibodies on the growth of HNSCC cells was studied. Soluble inhibitors of frizzled receptors have ben described to induce apoptosis secondary to their inhibition of frizzled signaling. Accordingly, this experiment was designed to test the efficacy of anti-Fz Abs to perform the same function.

Cell proliferation was determined by a colorimetric MTT-based assay. Briefly, either 7.5×10³ or 10×10³ SNU1076 cells per well were cultured in a 96 well plate. After 24 hour graded amounts of polyclonal goat anti-human frizzled-2 antibody containing 300 ng, 30 ng, 3 ng, and 0.3 ng were added in the culture medium. The same concentrations of goat serum or Goat antihuman IgG (Fisher Scientific, Pittsburgh, Pa.) were used as an isotype control. On 1, 2, 3, or 4 days after incubating antibody, 20 ul of MTT (3-[4,5-dimethylthiazol-2-yl]-2,5-diphenyl tetrazolium bromide)-based solution was added to the wells for four hours prior to lysis with 15% SDS, 0.015 M HCl. Absorbances at 570 and 650 nm were measured. The results are depicted in FIG. 4 and also given in Table IV below. Data represent the normalized growth fraction of the specific antibody treated cells to that of the control antibody treated cells (in triplicate).

TABLE IV
Cell proliferation in presence of anti-Fz Ab

| FZD | 300 ng | 30 ng | 3 ng | 0.3 ng |
|---|---|---|---|---|
| Day 1 | 87.88 ± 9.04 | 99.21 ± 9.07 | 108.68 ± 14.58 | 112.65 ± 13.50 |
| Day 2 | 68.50 ± 8.50 | 86.08 ± 10.80 | 90.33 ± 6.67 | 89.18 ± 7.97 |
| Day 3 | 65.09 ± 9.26 | 86.03 ± 5.74 | 75.14 ± 19.08 | 90.22 ± 2.64 |
| Day 4 | 53.82 ± 4.20 | 64.52 ± 7.41 | 88.19 ± 10.97 | 81.37 ± 7.07 |
| Day 5 | 53.75 ± 4.57 | 81.27 ± 9.04 | 92.98 ± 8.81 | 90.84 ± 5.71 |

As shown, treatment with antibodies markedly decreases the proliferation of SNU1076 cells. In a control experiment (results not shown), there was not appreciable effect of the same antibody on the growth of normal cells.

### Examle 4

### The Effects of Anti-Fz Abs on Cancer Cell Apoptosis

The effects of the anti-Fz Abs from Example 3 on apoptosis of SNU1076 cells was also studied. Cells were grown in RPMI-1640 supplemented with 10% FBS. The cells were treated for 72 hours with 300 ng/ml anti-Fz Ab, or control polyclonal antibodies. Two assays were used to quantify the cytotoxic effect of the antibodies as follows:

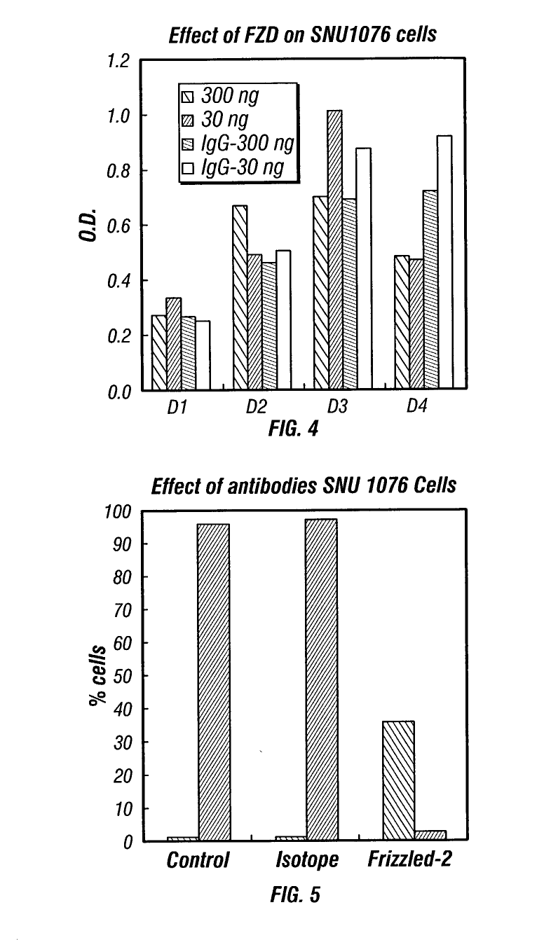

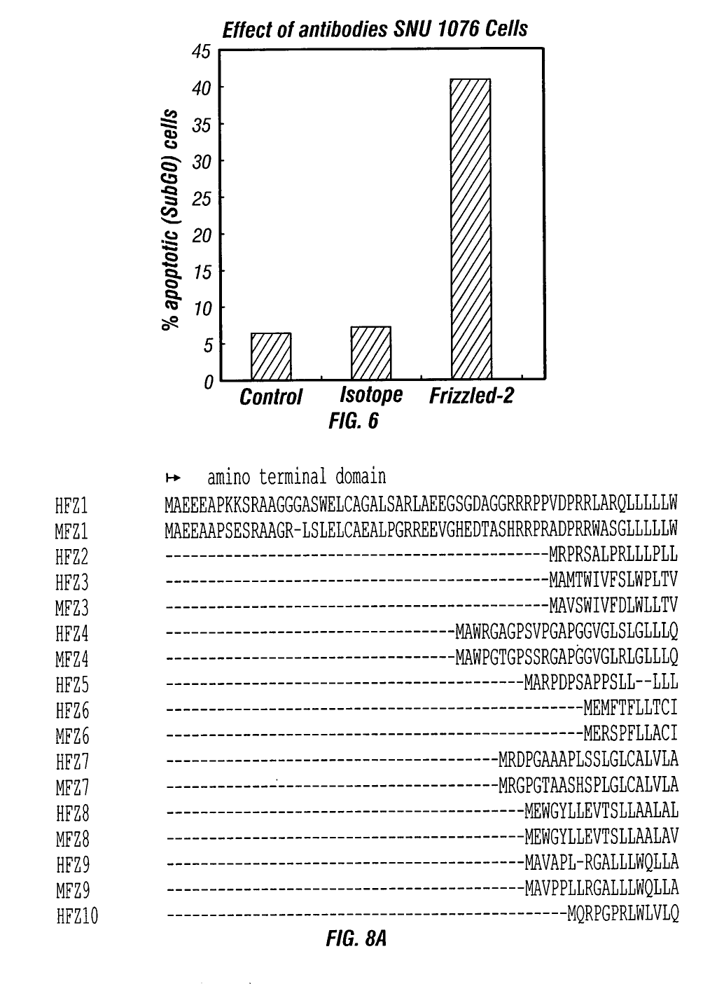

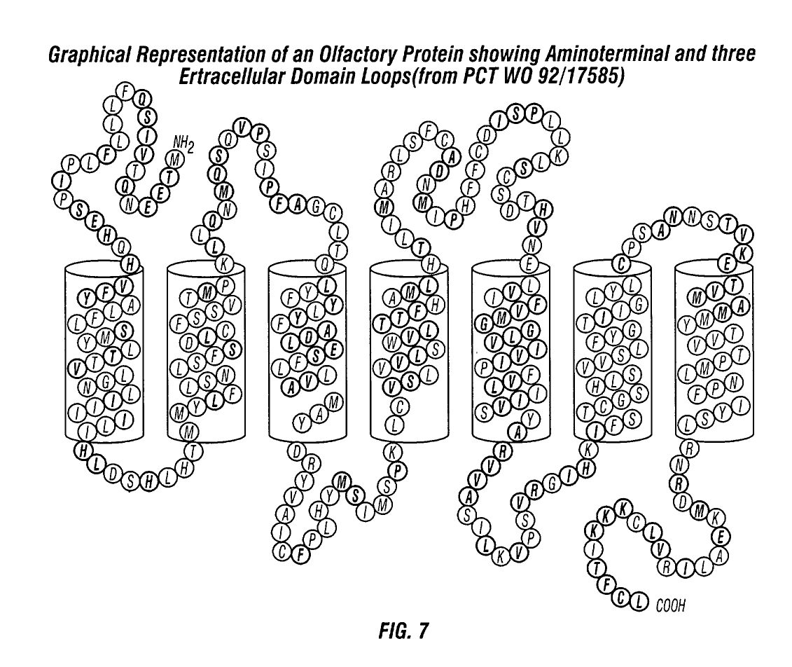

As shown in FIG. 5, cells were detached from the flasks by ftrysin treatment and incubated for 10 minutes in growing medium with 5 μg/ml Propidium iodide and 40 nM DiOC₆ and analyzed by flow cytometry. Viable cells (Alive, right bars) had high DiOC₆ (FL-1) and low PI (FL-3) faiorescence, while apoptotic cells (left bars) had low DiOC₆ (FL-1) and low PI (FL-3) fuiorescence.

As shown in FIG. 6, cells were detached from the flasks by try-psin treatment and incubated overnight in a hypotonic buffer (0.1% citrate, 0.1% SDS) containing 50 μg/ml PI and 100 μg/ml RNase. The amount of DNA was then measured by flow cytometry, and apoptotic cells were defined as having a DNA content lower than the G₀G₁ levels (sub-G₀ cells).

### Example 5

### Idenitification of tumor-specific frizzled epitopes

As described above in Examples 1 to 4, frizzled 2 antigens may be differentially overexpressed in cells of malignant phenotype, whereas many frizzled gene products may be expressed in normal and abnormal cells. Whereas the frizzled 2 systems is exemplary herein, it is readily apparent that tumor specific frizzled antigens from the other frizzled genes are equally attractive targets for cancer immunotherapies. Accordingly, the methods taught herein can easily be adapted to other frizzled genes and their protein products.

For example, a panel of tumor cells that can be screened are derived from the panel of 60 lines which are being characterized in the NIH Developmental Therapeutics Program. The cell lines that are currently available in the lab include: (non-small cell lung cancer) A549/ATCC, NCI-H226, NCI-H460, HOP-62, HOP-92,(colon cancer) HT29, HCT-116, (breast cancer) MCF7, NCI/ADR-RES, MDA-MB-231/ATCC, T-47D, (ovarian cancer) OVCAR-3, OVCAR-4, SK-OV-3, (leukemia) CCRF-CEM, K-562, MOLT-4, HL-60(TB), RPMI-8226, (renal cell) 786-0, TK-10, (prostate cancer) PC-3, DU-145. Normal control cell lines will be purchased as previously from Clonetics.

The expression of frizzled proteins can be confirmed with commercially available antibodies to frizzled isoforms, or where none are available, they can easily be prepared using known methods.

The overall strategy is to use the least conserved region of the frizzled protein, attempting to preserve the most native structure possible and to generate the most potent immune response. The most versatile method for designing vaccines of defined regions is naked plasmid DNA. The advantages are that the vectors can be rapidly redesigned to change the length of sequence that is expressed, discontinuous regions of the protein can be co-expressed, and the DNA sequence of the protein can be fused to other epitopes to enhance antigenicity. It affords the versatility of expressing soluble, membrane bound proteins, or small peptide fragments. Also gene transfer by this technique is a powerful tool to introduce multiple protein elements into the same or separate locations. In this system single or multiple proteins can be locally expressed. Injecting a combination of plasmids expressing antigens and costimulators like B7.1 and B7.2 results in enhanced immune responses.

Several plasmids have been constructed which are under the control of the cytomegalovirus (CMV) promoter which has been found to enable high levels of antigen expression in injected muscle. The pCMVint vector includes the cytomegalovirus (CMV) E1 promoter, the simian virus (SV40) t-intron, and the SV-40 polyadenylation site. The ACB vector has the same elements except the polyadenylation sequence is from the bovine growth hormone gene. For example, a preferred plasmid construct for frizzled-2 encodes the least homologous region of the frizzled gene between the ninth and tenth cysteine. These cysteines stabilize a configuration that enables antibody binding to the native protein. This polypeptide fragement is fused at the amino terminus or the carboxylterminus via a short linker to a tetanus toxin or measles MvF T helper epitope (see below). These minigenes are constructed with overlapping oligonucleotides. The oligonucleotides are 5′ prime phosphorylated with T4 kinase (Boehringer Mannheim, Indianapolis, Ind.) at room temperature for 30 miniutes, annealed by boiling an equimolar admixture of two complementary oligomers and slow cooling. The double stranded oligonucleotides are then ligated 3′ to the tissue plasminogen leader (TPA) leader into the EcoR47III site in frame and into the BamH1 site of the pBluescript SKII vector. The minigene is then subdloned into the pCMV and pACB vectors between the Pst1 and Xba1 sites as previously described.

The inserts for the vectors are designed as described above. The frizzled putative B cell epitope is from the published sequence. The tetanus toxin and measles MVF T helper epitopes have been optimized for human codon usage by the most frequently used codon per amino acid. The DNA constructs have an initiating methionine and stop codons added to the 5′ and 3′ ends respectively. The amino acid and DNA sequences are summarized below with the short GPSL linker sequence in bold and the T cell helper epitope underlined.

Tetanus toxin epitope fused to a frizzled domain

pFZD2-TT
Seq. ID 27:
MCVGQNHSEDGAPALLTTAPPPGLQPGAGGTPGGPGGGGAPPRYATLEHPFHC
-GPSL-VDDALINSTKIYSYFPSV-STOP
Seq. ID 28:
ATG TGC GTC GGC CAG AAC CAC TCC GAG GAC GGA GCT CCC GCG CTA CTC
ACC ACC GCG CCG CCG CCG GGA CTG CAG CCG GGT GCC GGG GGC ACC CCG
GGT GGC CCG GGC GGC GGC GGC GCT CCC CCG CGC TAC GCC ACG CTG GAG
CAC CCC TTC CAC TGC-GGC CCC AGC CTG-GTG GAC GAC GCC CTG ATC AAC
AGC ACC AAG ATC TAC AGC TAC TTT CCC AGC GTG TAG
pTT-FZD2
Seq. ID 29:
MVDDALINSTKIYSYFPSV-GPSL-
CVGQNHSEDGAPALLTTAPPPGLQPGAGGTPGGPGGGGAPPRYATLEHPFHC-STOP
Seq. ID 30:
ATG GTG GAC GAC GCC CTG ATC AAC AGC ACC AAG ATC TAC AGC TAC TTT
CCC AGC GTG-GGC CCC AGC CTG-TGC GTC GGC CAG AAC CAC TCC GAG GAC
GGA GCT CCC GCG CTA CTC ACC ACC GCG CCG CCG CCG GGA CTG CAG CCG
GGT GCC GGG GGC ACC CCG GGT GGC CCG GGC GGC GGC GGC GCT CCC CCG
CGC TAC GCC ACG CTG GAG CAC CCC TTC CAC TGC TAG

Measles MVF epitope fused to a frizzled domain

PFZD2-MMVF
Seq. ID 31:
MCVGQNHSEDGAPALLTTAPPPGLQPGAGGTPGGPGGGGAPPRYATLEHPFHC
GPSL-KLLSLIKGVIVHRLEGVE-STOP
Seq. ID 32:
ATG TGC GTC GGC CAG AAC CAC TCC GAG GAC GGA GCT CCC GCG CTA CTC
ACC ACC GCG CCG CCG CCG GGA CTG CAG CCG GGT GCC GGG GGC ACC CCG
GGT GGC CCG GGC GGC GGC GGC GCT CCC CCG CGC TAC GCC ACG CTG GAG
CAC CCC TTC CAC TGC-GGC CCC AGC CTG-AAG CTG CTG AGC CTG ATC AAG
GGC GTG ATC GTG CAC CGC CTG GAG GGC GTG GAG TAG
PMMVF-FZD2
Seq. ID 33:
MKLLSLIKGVIVHRLEGVE-GPSL-
CVGQNHSEDGAPALLTTAPPPGLQPGAGGTPGGPGGGGAPPRYATLEHPFHC-STOP
Seq. ID 34:
ATG AAG CTG CTG AGC CTG ATC AAG GGC GTG ATC GTG CAC CGC CTG GAG
GGC GTG GAG-GGC CCC AGC CTG-TGC GTC GGC CAG AAC CAC TCC GAG GAC
GGA GCT CCC GCG CTA CTC ACC ACC GCG CCG CCG CCG GGA CTG CAG CCG
GGT GCC GGG GGC ACC CCG GGT GGC CCG GGC GGC GGC GGC GCT CCC CCG
CGC TAC GCC ACG CTG GAG CAC CCC TTC CAC TGC TAG

Plasmid DNA is prepared using Qiagen Maxiprep (Chatsworth, Calif.) kits with the modification of adding one tenth volume 10% Triton X-114 (Sigma, St. Louis, Mo.) to the clarified bacterial lysate prior to applying it to a column. Prior to injection the residual endotoxin level is quantified using a limulus extract clot assay (Associates of Cape Cod, Woods Hole, Mass.). A level of ≦5 ng endotoxin/□g DNA need be obtained prior to use in an animal. The DNA is resuspended in a sterile pyrogen free saline solution for injection.

Twenty-eight female mice are divided into groups of 4 mice each. They are injected in the dermis of the tail with a combination of 50 μg plasmid encoding a costimulator and 50 μg linker plasmid diluted in normal saline at weeks zero, one and two. A group with empty vector is included as a negative control. The groups are as follows:

TABLE V
Vector groups for expresssion of frizzled-2 receptors

| Group | Plasmid 1 | Plasmid 2 |
|---|---|---|
| A | pTT-FZD2 | nCMV |
| B | pTT-FZD2 | nCMVB7-1 |
| C | pTT-FZD2 | nCMVB7-2 |
| D | pFZD2-TT | nCMV |
| E | pFZD2-TT | nCMVB7-1 |
| F | pFZD2-TT | nCMVB7-2 |
| G | — | nCMV |

Another group of mice in similar groups is immunized using the pMMVF-FZD2 and pFZD2-MMVF set of linked epitope plasmids. ). The nCMVB7-1 and nCMVB7-2 constructs encode the cDNAs for murine CD80 and CD86 (provided by G. Freeman (Dana-Farber Cancer Institute, Boston, Mass.).

Mice are bled prior to the start of the experiment and then every two weeks thereafter. Serum is separated and stored at −20° C. prior to testing. On week ten (seven weeks after the last injection) mice are sacrificed. The titers of antibody are tested by anti-peptide ELISA. Ninety-six well plates (Costar)are coated with 50 ul/ well 20 μg/ml peptide in phosphate buffered saline (PBS) overnight at 4° C. The plates are then washed and blocked with 200 ul/ well 2% bovine serum albumin (BSA) in PBS. Sera are diluted in 2% BSA in PBS. After overnight incubation at 4° C. the plates are washed. Bound murine IgG is detected by alkaline phosphatase conjugated-goat anti-murine IgG (Jackson lmmunoresearch Laboratories) followed by p-nitrophenylphosphate substrate. The titration curves for each sera are compared using DeltaSOFT II v. 3.66 (Biometallics, Princeton, N.J.).

Mice that develop sufficiently high titers of antibody that bind to the peptide are tested for specificity to frizzled 2 by fluorescent cytometry with cells that express the protein by transfection and known tumor cells that have the mRNA. Binding is tested by Western blot analysis of cells that express this isoform and to cells that have been found to express other frizzled family members.

If the antibody response is weak then the vectors can be redesigned with other known potent T helper epitopes. In addition, other vectors can be designed where the frizzled protein fragment is altered to achieve the most desirable conformation. Another immunization strategy will be to use a prime boost method. The animals are originally injected with plasmid DNA and then are boosted with peptide or recombinant protein in incomplete Freund's adjuvant.

Once antibodies have been identified that delay cancer cell growth in cell culture, the ability of these antibodies can be tested for potential in vivo efficacy in mice. For example, the H-2^{b} thymoma line EL4 can be used as a syngeneic tumor in C57B1/6 mice. This line is transfected with a human frizzled expression vector and selected in neomycin. The expression vector is made by excising the frizzled containing insert from a pET3a bacterial expression vector with Nde1 and BamH1 and ligating the insert into pcDNA3 which has a CMV promoter and a neomycin selection cassette. Thirty two female C57B1/6 mice are divided into groups of 8 mice each. They are injected in the dermis of the tail with a combination of 50 μg plasmid encoding a costimulator and 50 μg linker plasmid diluted in normal saline at weeks zero, one and two. A group with empty vector is included as a negative control. On day 28 the mice are injected with 20×10⁶ frizzled transfected EL4 cells or untransfected cells. The mice are monitored three times a week for weight, and tumor growth measured with a caliper. Tumor volume is calculated by lengthxwidth²×π/6. Mice are sacrificed four weeks post tumor challenge or if the tumor burden reaches approximately 2000 mm³. Inhibition of tumor growth is determined by ANOVA.

Polyclonal antibodies may have low levels of cross reactivity with other proteins that are below the detection level of the binding assays but convey a biologic effect. The antibodies may have not only a blocking or a steric effect, but may also be able to cross link the receptor and make it constitutively active. The presence of the effector antibody may be a minor population in the polyclonal sera and the effect may appear insignificant. Whereas a monoclonal would have a pure population and only one effect. However the assay using polyclonal antibodies will determine if the frizzled expressing cell lines are susceptible to anti-proliferative activity in the pool of anti-frizzled IgG. This provides useful information with respect to the methods that are useful for screening panels of monoclonal antibodies.

The examples set forth above are provided to give those of ordinary skill in the art with a complete disclosure and description of how to make and use the preferred embodiments of the compositions, and are not intended to limit the scope of what the inventors regard as their invention. Modifications of the above-described modes for carrying out the invention that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

We claim:

1. A purified antibody for modulating a biological activity of a malignant cell that expresses a frizzled receptor, wherein said antibody specifically binds to at least one epitope in an extracellular domain of the frizzle receptor expressed on the malignant cell.

2. The purified antibody of claim 1, wherein the extracellular domain comprises an amino terminal peptide fragment of the frizzled receptor.

3. The purified antibody of claim 1 further comprising an antibody fragment having an antigen binding region that specifically binds to the epitope.

4. The purified antibody of claim 1, wherein the antibody is capable of sensitizing malignant cells expressing the frizzled receptor to a cytotoxic factor.

5. The purified antibody of claim 1, wherein the antibody inhibits binding of a Wnt ligand to the frizzled receptor.

6. The purified antibody of claim 1 further comprising a detectable label.

7. The purified antibody of claim 1, wherein the antibody is a human antibody.

8. The purified antibody of claim 1, wherein the antibody is a monoclonal antibody.

9. The purified antibody of claim 1, wherein the antibody binds to a frizzled-2 receptor amino terminal extracellular domain.

10. The purified antibody of claim 1, wherein the frizzled receptor amino terminal extracellular domain has a sequence that is greater than 80% homologous to an amino acid sequence selected from the group Seq. ID No.s 61, 63, 64, 66, 68, 69, 71, 73, 75 and 77.

11. An isolated nucleic acid, comprising at least one nucleotide fragment encoding a peptide having an amino acid sequence that is greater than 80% homologous to an amino acid sequence selected from the group Seq. ID No.s 61, 63, 64, 66, 68, 69, 71, 73, 75 and 77.

12. The isolated nucleic acid of claim 11, further comprising at least one nucleotide fragment encoding a T cell epitope .

13. A transgenic non-human animal, comprising at least one isolated nucleic acid of claim 11.

14. A recombinant vector, comprising at least one nucleic acid according to claim 11 functionally attached to a promoter region upstream of the nucleic acid.

15. A host cell comprising at least one recombinant vector according to claim 14.

16. A pharmaceutical composition comprising a purified antibody for modulating a biological activity of a malignant cell that expresses a frizzled receptor, wherein said antibody specifically binds to at least one epitope in an extracellular domain of the frizzle receptor expressed on the malignant cell, in a pharmaceutically acceptable carrier.

17. A method for modulating a biological activity of a malignant cell that expresses a frizzled receptor comprising administering a pharmaceutical composition comprising a purified antibody for modulating a biological activity of a malignant cell that expresses a frizzled receptor, wherein said antibody specifically binds to at least one epitope in an extracellular domain of the frizzle receptor expressed on the malignant cell, in a pharmaceutically acceptable carrier.

18. A frizzled receptor epitope conjugate comprising at least one epitope in an extracellular domain of the frizzle receptor expressed on a malignant cell and at least one epitope specific to a T cell antigen.

19. The conjugate of claim 18, wherein the T cell antigen is also an epitope in an extracellular domain of the frizzle receptor expressed on a malignant cell

20. The conjugate of claim 18 further comprising a linker moiety.

21. The conjugate of claim 20, wherein the linker is GPSL.

22. A pharmaceutical composition useful as a vaccine against malignancy for administration to a patient having a predisposition for the malignancy, comprising a purified antibody for modulating a biological activity of a malignant cell that expresses a frizzled receptor, wherein said antibody specifically binds to at least one epitope in an extracellular domain of the frizzle receptor expressed on the malignant cell.

23. A method of immunizing a subject against a malignancy comprised of malignant cells that express a frizzled receptor, said method comprising the steps of:
a) identifying an antibody for modulating a biological activity of the malignant cell that expresses a frizzled receptor, wherein said antibody specifically binds to at least one epitope in an extracellular domain of the frizzle receptor expressed on the malignant cell; and
b) administering the antibody in a pharmaceutically acceptable carrier in an amount sufficient to inhibit the malignancy.

24. A method of treating a subject with a malignancy comprised of malignant cells that express a frizzled receptor, said method comprising the steps of::
a) identifying an antibody for modulating a biological activity of the malignant cell that expresses a frizzled receptor, wherein said antibody specifically binds to at least one epitope in an extracellular domain of the frizzle receptor expressed on the malignant cell; and
b) administering the antibody in a pharmaceutically acceptable carrier in an amount sufficient to modulate a biological activity of the malignant cell.

25. An assay for identifying a frizzled receptor expressed by a malignant cell, wherein said frizzled receptor comprises at least one epitope in an extracellular domain, comprising the steps of:
a) identifying an antibody that specifically binds to the epitope;
b) exposing a sample of cells suspected of expressing the frizzled receptor to the antibody; and
c) determining the extent of binding of the antibody to the cells.

26. A screening assay for identification of small molecules that modulate frizzled receptor activity, comprising:
a) selecting a library of the small molecules comprising a plurality of different chemical structures;
b) contacting the small moleucles with an extracellular domain of a frizzled receptor which is capable of binding to its corresponding Wnt protein; and
c) measuring binding of a ligand to the frizzled receptor in the presence of the small molecule, wherein the ligand is selected from the group consisting of the small molecule, the Wnt protein, and an antibody to the extracellular domain of the frizzled receptor.
